(19) United States
(12) Patent Application Publication
(10) Pub. No.: US 2015/0004252 A1
(43) Pub. Date: Jan. 1, 2015

(54) METHOD FOR PREDICTING THE CLINICAL RESPONSE TO CHEMOTHERAPY IN A SUBJECT WITH CANCER

(71) Applicant: Juan Carlos Lacal SanJuan, Madrid (ES)

(72) Inventor: Juan Carlos Lacal SanJuan, Madrid (ES)

(73) Assignee: TRASLATIONAL CANCER DRUGS PHARMA, S.L, Valladolid (ES)

(21) Appl. No.: 14/128,369

(22) PCT Filed: Jun. 20, 2012
(86) PCT No.: PCT/EP2012/061790

§ 371 Date: Jul. 31, 2014

Related U.S. Application Data
(60) Provisional application No. 61/507,833, filed on Jul. 14, 2011.

(30) Foreign Application Priority Data
Jun. 20, 2011 (EP) 11382211.8
Sep. 16, 2011 (EP) 11382294.4

(51) Int. Cl. C12Q 1/68; A61K 33/24; A61K 31/4709; G01N 33/574

(52) U.S. Cl. CPC C12Q 1/6886; G01N 33/57496; A61K 33/24; A61K 31/4709; G01N 33/57423; C12Q 2600/158; C12Q 2600/106; G01N 2333/91215
USPC 424/649; 435/6.12; 435/7.4; 514/313

(57) ABSTRACT

The invention relates to the use of choline kinase alpha as predictive marker for the determination of the response to a chemotherapeutic treatment in a subject suffering from cancer, particularly for predicting the clinical response of a subject suffering from non-small cell lung cancer to a platinum-based chemotherapeutic treatment. The invention relates to methods for designing a personalised therapy for subjects suffering from cancer, particularly from non-small cell lung cancer, based on the expression levels of choline kinase alpha as well as to methods for the treatment of non-small cell lung cancer using a platinum-based chemotherapeutic treatment based in a subject wherein the subject is selected based on the expression levels of choline kinase alpha.

### FIELD OF THE INVENTION

The invention relates to the field of diagnostics and, more in particular, to a method for predicting the clinical response of a subject suffering from cancer to a chemotherapeutic treatment, particularly for predicting the clinical response of a subject suffering from non-small cell lung cancer to a platinum-based chemotherapeutic treatment, based on the expression levels of ChoKα gene in a sample from said subject. The invention also relates to a method for designing an individual therapy for a subject suffering from said disease as well as to a method for selecting patient likely to respond to a given therapy.

### BACKGROUND OF THE INVENTION

Routine cancer management using chemotherapy, whether as definitive or adjuvant therapy, has improved patient's absolute survival when compared with non-chemotherapy control. However, not all the chemotherapeutic treatments available are suitable for all patients. The efficacy of chemotherapeutic drugs in patients suffering from cancer is influenced by the presence of certain genetic markers. Patients whose tumours have low probability to respond to a chemotherapeutic treatment may omit chemotherapy altogether or may be candidates for alternative treatments, avoiding unnecessary therapeutic side effects.

Therefore, there is a necessity for a personalized approach for the treatment of the disease, particularly in cancers such as lung cancer, colon cancer, melanoma, pancreas cancer, prostate cancer, glioma, bladder cancer, ovarian cancer, hepatobiliary cancer, breast cancer and lymphomas.

Lung cancer is one of the leading causes of worldwide death, and non-small cell lung cancer (NSCLC) accounts for approximately 85% of all lung cancers, with 1.2 million new cases worldwide each year. NSCLC resulted in more than one million deaths worldwide in 2001 and is the leading cause of cancer-related mortality in both men and women (31% and 25%, respectively).

The prognosis of advanced NSCLC is dismal. A recent Eastern Cooperative Oncology Group trial of 1155 patients showed no differences among the chemotherapies used: cisplatin/paclitaxel, cisplatin/gemcitabine, cisplatin/docetaxel and carboplatin/paclitaxel. Overall median time to progression was 3.6 months, and median survival was 7.9 months.

The five-year survival rate varies according to the TNM classification of Malignant Tumours. TNM is a cancer staging system that describes the extent of cancer in a patient's body based on the extent of the tumour (T), the extent of spread to the lymph nodes (N) and the presence of metastasis (M). A study which took place at the Mayo Clinic showed that the estimated overall five-year survival rates of patients with non-small cell lung cancer (NSCLC) by disease stage was 66% for pathologic stage IA, 53% for stage IB, 42% for stage IIA, 36% for stage IIB, 10% for stage IIIA, 12% for stage IIIB and 4% for stage IV (Yang P., et al. 2005. Chest, 128:452-462).

About 70% of NSCLC cases are advanced at diagnosis and are always treated with chemotherapy. Platinum-based combinations with newer agents have been widely accepted as the first-line treatment of advanced NSCLC, but the frequent development of platinum-resistance is a major obstacle for treatment of these patients at present. Furthermore, there are still many patients who receive chemotherapy from which they do not benefit, typically experiencing unnecessary toxicity and, a negative impact on their quality of life. Therefore, the advent of the predictive value of any new biomarker is essential in order to improve the patient's outcome by supporting the fitting of NSCLC patients with the most effective personalized anticancer treatment available.

An attempt to provide reliable markers for the response of lung cancer patients to platinum-based chemotherapy has been performed by Lord et al., (Clin. Cancer Res., 2002, 8:2286-2291) and Ceppi P. et al., (Ann Oncol., 2006, 17:1818-1825) (using ERCC as a marker); Davidson et al., (Cancer Res., 2004, 64:3761-3766) and Rosell et al., (Clin. Cancer Res., 2004, 10:1318-1325) (using ribonucleotide reductase large subunit 1 as a marker); Ceppi P. et al., (Ann Oncol., 2006, 17:1818-1825) (using ribonucleotide reductase M1 subunit as a marker); Ceppi, P. et al., (Clin Cancer Res., 2009, 15:1039-45) (using DNA polymerase eta as a marker) and Taron et al., (Hum. Mol. Genetics, 2004, 13:2443-2449) (using BRCA1 as a marker).

However, there is still a need for further markers useful for predicting the response of cancer patients to chemotherapeutic treatment, particularly for predicting the response of NSCLC lung cancer patients to platinum-based chemotherapeutic treatment.

### BRIEF DESCRIPTION OF THE INVENTION

In a first aspect, the invention relates to an in vitro method for predicting the clinical response of a subject suffering from cancer to a chemotherapeutic treatment comprising determining the expression level of the choline kinase alpha (ChoKα) gene in a sample from the subject.

In another aspect, the invention relates to an in vitro method for designing an individual therapy for a subject suffering from cancer comprising determining the expression levels of the choline kinase alpha (ChoKα) gene in a sample from the subject.

In yet another aspect, the invention relates to the use of a reagent capable of determining the expression levels of the ChoKα gene in a sample from a subject suffering from cancer for predicting the clinical response of said subject to a chemotherapeutic treatment and for designing an individual therapy for a subject suffering from said cancer.

In yet another aspect, the invention relates to a platinum-based chemotherapeutic treatment for use in the treatment of NSCLC in a subject, wherein a sample of said subject shows low or substantially the same expression levels of the ChoKα gene with respect to reference values.

In yet another aspect, the invention relates to a ChoKα inhibitor, a folate antimetabolite, an EGFR-targeted drug or a combination of one or more of the above for use in the therapy of a subject suffering from NSCLC, wherein a sample of said subject shows high expression levels of the ChoKα gene with respect to reference values.

### BRIEF DESCRIPTION OF THE FIGURE

FIG. 1 shows the Kaplan-Meier plots for ChoKα expression and progression-free survival in subjects with advanced NSCLC treated with platinum-based chemotherapeutic treatment.

### DETAILED DESCRIPTION OF THE INVENTION

The inventors of the present invention have discovered that, surprisingly, the expression levels of the ChoKα gene are also useful for predicting the response to a chemotherapeutic treatment in subjects suffering from cancer, particularly for predicting the response to a platinum-based chemotherapeutic treatment in subjects suffering from non-small cell lung cancer (NSCLC). In this sense, high expression levels of the ChoKα gene correlate with poor response to platinum-based chemotherapy of the subject suffering from NSCLC. Based on these findings, the inventors have developed the methods of the present invention in their different embodiments that will be described now in detail.

The results provided in the example of the present invention clearly show a significant association of ChoKα expression with failure to respond to platinum-based chemotherapy. Thus, these results suggest that the prognosis of subjects with high expression of ChoKα would be poor after chemotherapy with platinum, which plays a central role in the management of NSCLC.

### Method for Predicting the Clinical Outcome of a Cancer Patient

In one aspect, the invention relates to an in vitro method (hereinafter first method of the invention) for predicting the clinical response of a subject suffering from cancer to a chemotherapeutic treatment comprising determining the expression levels of the choline kinase alpha (ChoKα) gene in a sample from the subject.

The term “predicting”, as used herein, refers to the determination of the likelihood that the subject suffering from cancer will respond either favorably or unfavorably to a given therapy. Especially, the term “prediction”, as used herein, relates to an individual assessment of the expected response of a subject suffering from cancer if the tumour is treated with a given therapy. In a preferred embodiment, the term “predicting” refers to the determination of the likelihood that a subject suffering from NSCLC will respond either favorably or unfavorably to a given therapy.

The term “clinical response”, as used herein, refers to the response of the subject suffering from cancer to a chemotherapeutic treatment. In a preferred embodiment the “clinical response” refers to the response of the subject suffering from NSCLC to a therapy with a platinum-based chemotherapeutic treatment. Standard criteria (Miller, et al. Cancer, 1981; 47:207-14) that can be used herewith to evaluate the response to chemotherapy include response, stabilization and progression.

The term “response”, as used herein, can be a complete response (or complete remission) which is the disappearance of all detectable malignant disease or a partial response which is defined as approximately >50% decrease in the sum of products of the largest perpendicular diameters of one or more lesions (tumour lesions), no new lesions and no progression of any lesion. Subjects achieving complete or partial response were considered “responders”, and all other subjects were considered “non-responders”. As will be understood by those skilled in the art, such an assessment is usually not intended to be correct for all (i.e. 100 percent) of the subjects to be identified. The term, however, requires that a statistically significant portion of subjects can be identified (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley and Sons, New York 1983. Preferred confidence intervals are at least 90 percent, at least 95 percent, at least 97 percent, at least 98 percent or at least 99 percent. The p-values are, preferably, 0.1, 0.05, 0.01, 0.005, or 0.0001. More preferably, at least 60 percent, at least 70 percent, at least 80 percent or at least 90 percent of the subjects of a population can be properly identified by the method of the present invention.

The term “stabilization”, as used herein, is defined as a <50% decrease or a <25% increase in tumour size.

The term “progression”, as used herein, is defined as an increase in the size of tumour lesions by >25% or appearance of new lesions.

Any other parameter which is widely accepted for comparing the efficacy of alternative treatments can be used for determining a response to treatment and include, without limitation:
disease-free progression which, as used herein, describes the proportion of subjects in complete remission who have had no recurrence of disease during the time period under study.
disease-free survival (DFS), as used herewith, is understood as the length of time after treatment for a disease during which a subject survives with no sign of the disease.
objective response which, as used in the present invention, describes the proportion of treated subjects in whom a complete or partial response is observed.
tumour control which, as used in the present invention, relates to the proportion of treated subjects in whom complete response, partial response, minor response or stable disease ≧6 months is observed.
progression free survival which, as used herein, is defined as the time from start of treatment to the first measurement of cancer growth.
Time to progression (TTP), as used herein, relates to the time after a disease is treated until the disease starts to get worse. The term “progression” has been previously defined.
six-month progression free survival or “PFS6” rate which, as used herein, relates to the percentage of subjects wherein free of progression in the first six months after the initiation of the therapy and
median survival which, as used herein, relates to the time at which half of the subjects enrolled in the study are still alive.

In a particular embodiment of the first method of the invention, the clinical response is measured as time to progression or a progression-free survival.

The term “subject”, as used herein, refers to all animals classified as mammals and includes, but is not restricted to, domestic and farm animals, primates and humans, e.g., human beings, non-human primates, cows, horses, pigs, sheep, goats, dogs, cats or rodents. Preferably, the subject is a male or female human of any age or race. In the context of the present invention, the subject is a subject suffering from cancer or previously diagnosed with cancer, preferably is a subject suffering from NSCLC or previously diagnosed with NSCLC.

The terms “cancer” and “tumour” refer to the physiological condition in mammals characterized by unregulated cell growth. The methods of the present invention are useful in any cancer or tumour, such as, without limitation, breast, heart, lung, small intestine, colon, spleen, kidney, bladder, head, neck, ovarian, prostate, brain, pancreas, skin, bone, bone marrow, blood, thymus, uterus, testicles, hepatobiliary and liver tumours. In particular, tumours whose chemotherapeutic response may be predicted with the methods of the invention include adenoma, angiosarcoma, astrocytoma, epithelial carcinoma, germinoma, glioblastoma, glioma, hemangioendothelioma, hemangiosarcoma, hematoma, hepatoblastoma, leukaemia, lymphoma, medulloblastoma, melanoma, neuroblastoma, hepatobiliary cancer, osteosarcoma, retinoblastoma, rhabdomyosarcoma, sarcoma, and teratoma. In particular, the tumour/cancer is selected from the group of acral lentiginous melanoma, actinic keratosis adenocarcinoma, adenoid cystic carcinoma, adenomas, adenosarcoma, adenosquamous carcinoma, astrocytic tumours, bartholin gland carcinoma, basal cell carcinoma, bronchial gland carcinoma, capillary carcinoid, carcinoma, carcinosarcoma, cholangiocarcinoma, cystadenoma, endodermal sinus tumour, endometrial hyperplasia, endometrial stromal sarcoma, endometrioid adenocarcinoma, ependymal sarcoma, Swing's sarcoma, focal nodular hyperplasia, germ cell tumours, glioblastoma, glucagonoma, hemangioblastoma, hemangioendothelioma, hemangioma, hepatic adenoma, hepatic adenomatosis, hepatocellular carcinoma, hepatobiliary cancer, insulinoma, intraepithelial neoplasia, interepithelial squamous cell neoplasia, invasive squamous cell carcinoma, large cell carcinoma, leiomyosarcoma, melanoma, malignant melanoma, malignant mesothelial tumour, medulloblastoma, medulloepithelioma, mucoepidermoid carcinoma, neuroblastoma, neuroepithelial adenocarcinoma, nodular melanoma, osteosarcoma, papillary serous adenocarcinoma, pituitary tumours, plasmacytoma, pseudosarcoma, pulmonary blastoma, renal cell carcinoma, retinoblastoma, rhabdomyosarcoma, sarcoma, serous carcinoma, small cell carcinoma, soft tissue carcinoma, somatostatin-secreting tumour, squamous carcinoma, squamous cell carcinoma, undifferentiated carcinoma, uveal melanoma, verrucous carcinoma, vipoma, Wilm's tumour. Even more preferably, the tumour/cancer include intracerebral cancer, head and neck cancer, rectal cancer, astrocytoma, glioblastoma, small cell cancer, and non-small cell cancer, preferably non-small cell lung cancer, metastatic melanoma, androgen-independent metastatic prostate cancer, androgen-dependent metastatic prostate cancer and breast cancer. In a preferred embodiment the cancer is selected from lung cancer, colon cancer, melanoma, pancreatic cancer, prostate cancer, glioma, bladder cancer, ovarian cancer, hepatobiliary cancer, breast cancer and lymphoma. In a more preferred embodiment the cancer is lung cancer, preferably non-small cell lung cancer (NSCLC).

The term non-small cell lung cancer (NSCLC), as used herein, refers to a group of heterogeneous diseases grouped together because their prognosis and management is roughly identical and includes, according to the histologic classification of the World Health Organization/International Association for the Study of Lung Cancer (Travis W D et al. Histological typing of lung and pleural tumours. 3^{rd} ed. Berlin: Springer-Verlag, 1999):
(i) squamous cell carcinoma (SCC), accounting for 30% to 40% of NSCLC, starts in the larger breathing tubes but grows slower meaning that the size of these tumours varies on diagnosis.
(ii) adenocarcinoma is the most common subtype of NSCLC, accounting for 50% to 60% of NSCLC, which starts near the gas-exchanging surface of the lung and which includes a subtype, the bronchioalveolar carcinoma, which may have different responses to treatment.
(iii) large cell carcinoma is a fast-growing form that grows near the surface of the lung. It is primarily a diagnosis of exclusion, and when more investigation is done, it is usually reclassified to squamous cell carcinoma or adenocarcinoma.
(iv) adenosquamous carcinoma is a type of cancer that contains two types of cells: squamous cells (thin, flat cells that line certain organs) and gland-like cells.
(v) carcinomas with pleomorphic, sarcomatoid or sarcomatous elements. This is a group of rare tumours reflecting a continuum in histologic heterogeneity as well as epithelial and mesenchymal differentiation.
(vi) carcinoid tumour is a slow-growing neuroendocrine lung tumour and begins in cells that are capable of releasing a hormone in response to a stimulus provided by the nervous system.
(vii) carcinomas of salivary gland type begin in salivary gland cells located inside the large airways of the lung.
(viii) unclassified carcinomas include cancers that do not fit into any of the aforementioned lung cancer categories.

In a preferred embodiment, the NSCLC is selected from squamous cell carcinoma of the lung, large cell carcinoma of the lung and adenocarcinoma of the lung.

The predictive method according to the present invention allows the determination of the clinical response of a subject suffering from cancer to a chemotherapeutic treatment in patients having different stages of NSCLC, including patients in with Stage I NSCLC, stage II NSCLC, stage III NSCLC and stage IV NSCLC. Stages I, II, III and IV in lung cancer are defined as follows.

The term “stage I NSCLC”, as used herein, refers to tumor which is present in the lungs but the cancer has not been found in the chest lymph nodes or in other locations outside of the chest. Stage I NSCLC is subdivided into stages IA and IB, usually based upon the size of the tumor or involvement of the pleura, which is lining along the outside of the lung. In Stage IA, the tumor is 3 centimeters (cm) or less in size and has invaded nearby tissue minimally, if at all. The cancer has not spread to the lymph nodes or to any distant sites. In Stage IB, the tumor is more than 3 cm in size, has invaded the pleural lining around the lung, or has caused a portion of the lung to collapse. The cancer has not spread to the lymph nodes or to any distant sites. Stage IA corresponds to stages T1N0M0 of the TNM classification. Stage IB corresponds to T2M0N0 of the TNM classification.

The term “Stage II NSCLC”, as used herein, refers to a cancer which has either begun to involve the lymph nodes within the chest or has invaded chest structures and tissue more extensively. However, no spread can be found beyond the involved side of the chest, and the cancer is still considered a local disease. Stage II is subdivided into stages IIA and IIB. Stage IIA refers to tumors which are 3 cm or smaller and has invaded nearby tissue minimally, if at all. One or more lymph nodes on the same side of the chest are involved, but there is no spread to distant sites. Stage IIB is assigned in two situations: when there is a tumor larger than 3 cm with some invasion of nearby tissue and involvement of one or more lymph nodes on the same side of the chest; or for cancers that have no lymph node involvement, but have either invaded chest structures outside the lung or are located within 2 cm of the carina (the point at which the trachea, or the tube that carries air to the lungs, splits to reach the right and left lungs). Stage IIA corresponds to T1N1M0 or T2N1M0 of the TNM classification. Stage BB correspond to T3N0M0 according to the TNM classification.

The term “Stage III NSCLC”, as used herein, refers to tumors which have invaded the tissues in the chest more extensively than in stage II, and/or the cancer has spread to lymph nodes in the mediastinum. However, spread (metastasis) to other parts of the body is not detectable. Stage III is divided into stages IIIA and IIIB Stage IIIA refers to a single tumor or mass that is not invading any adjacent organs and involves one or more lymph nodes away from the tumor, but not outside the chest. Stage IIIB refers to a cancer which has spread to more than one area in the chest, but not outside the chest. Stage IIIA corresponds to T1N2M0, T2N2M0, T3N1M0, T3N2M0, T4N0M0 or T4N1M0 according to the TNM classification. Stage IIIB corresponds to T1N3M0, T2N3M0, T3N3M0, T4N2M0 or T4N3M0 according to the TNM classification.

The term “Stage IV NSCLC”, as used herein, refers to a cancer which has spread, or metastasized, to different sites in the body, which may include the liver, brain or other organs. Stage IV corresponds to any T or any N with M1.

The TNM classification is a staging system for malignant cancer. As used herein the term “TNM classification” refers to the 6^{th} edition of the TNM stage grouping as defined in Sobin et al. (International Union Against Cancer (UICC), TNM Classification of Malignant tumors, 6^{th} ed. New York; Springer, 2002, pp. 191-203) (TNM6) and AJCC Cancer Staging Manual 6th edition; Chapter 19; Lung-original pages 167-177 whereby the tumors are classified by several factors, namely, T for tumor, N for nodes, M for metastasis as follows

T: Primary tumor cannot be assessed, or tumor proven by the presence of malignant cells in sputum or bronchial washings but not visualized by imaging or bronchoscopy:
T0 No evidence of primary tumor,
Tis Carcinoma in situ,
T1 Tumor 3 cm or less in greatest dimension, surrounded by lung or visceral pleura, without bronchoscopic evidence of invasion more proximal than the lobar bronchus (for example, not in the main bronchus),
T2 Tumor more than 3 cm but 7 cm or less or tumor with any of the following features (T2 tumors with these features are classified T2a if 5 cm or less): involves main bronchus, 2 cm or more distal to the carina; invades visceral pleura (PL1 or PL2); associated with atelectasis or obstructive pneumonitis that extends to the hilar region but does not involve the entire lung,
T3: Tumor more than 7 cm or one that directly invades any of the following: parietal pleural (PL3), chest wall (including superior sulcus tumors), diaphragm, phrenic nerve, mediastinal pleura, parietal pericardium; or tumor in the main bronchus less than 2 cm distal to the carinal but without involvement of the carina; or associated atelectasis or obstructive pneumonitis of the entire lung or separate tumor nodule(s) in the same lobe and
T4 Tumor of any size that invades any of the following: mediastinum, heart, great vessels, trachea, recurrent laryngeal nerve, esophagus, vertebral body, carina, separate tumor nodule(s) in a different ipsilateral lobe.

### N (Regional Lymph Nodes):

NX Regional lymph nodes cannot be assessed
N0 No regional lymph node metastases
N1 Metastasis in ipsilateral peribronchial and/or ipsilateral hilar lymph nodes and intrapulmonary nodes, including involvement by direct extension
N2 Metastasis in ipsilateral mediastinal and/or subcarinal lymph node(s)
N3 Metastasis in contralateral mediastinal, contralateral hilar, ipsilateral or contralateral scalene, or supraclavicular lymph node(s)
   M: Distant metastasis
M0 No distant metastasis
M1 Distant metastasis

In a preferred embodiment, the NSCLC is advanced stage NSCLC. In yet another embodiment, the NSCLC is stage IIIA, IIIB or IV NSCLC.

As previously explained, the first method of the invention allows the skilled person to predict the clinical response of a subject suffering from cancer to a chemotherapeutic treatment.

The term “treat” or “treatment” refers to a therapeutic treatment, as well as a prophylactic or prevention method, wherein the goal is to prevent or reduce an unwanted physiological change or disease, such as cancer. Beneficial or desired clinical results include, but not limiting, release of symptoms, reduction of the length of the disease, stabilized pathological state (specifically not deteriorated), retardation in the disease's progression, improve of the pathological state and remission (both partial and total), both detectable and not detectable. “Treatment” can mean also to prolong survival, compared to the expected survival if the treatment is not applied. Those who need the treatment include those who are suffering from cancer, as well as those with tendency to suffer from cancer. In a preferred embodiment, those who need the treatment include those who are suffering from NSCLC, as well as those with tendency to suffer from NSCLC.

In the context of the present invention, a “chemotherapeutic treatment” refers to a treatment with an antineoplastic drug used to treat cancer or the combination of more than one of these drugs into a cytotoxic standardized treatment regimen. In the context of the present invention, the term “chemotherapeutic treatment” comprises any antineoplastic agent including small sized organic molecules, peptides, oligonucleotides and such like used to treat any kind of cancer as well as related processes such as angiogenesis or metastasis. Drugs included in the definition of chemotherapy are, without limitation, alkylating agents such as nitrogen mustards/oxazaphosphorines (e.g. cyclophosphamide, ifosfamide), nitrosoureas (e.g. carmustine), triazenes (e.g. temozolamide), and alkyl sulfonates (e.g. busulfan); anthracycline antibiotics such as doxorubicin and daunorubicin, taxans such as Taxol™ and docetaxel, vinca alkaloids such as vincristin and vinblastine, 5-fluorouracil (5-FU), leucovorin, irinotecan, idarubicin, mitomycin C, oxaliplatin, raltitrexed, pemetrexed, tamoxifen, cisplatin, carboplatin, methotrexate, actinomycin D, mitoxantrone, blenoxane, mithramycin, methotrexate, paclitaxel, 2-methoxyestradiol, prinomastat, batimastat, BAY 12-9566, carboxyamidotriazole, CC-1088, dextromethorphan acetic acid, dimethylxanthenone acetic acid, endostatin, IM-862, marimastat, penicillamine, PTK787/ZK 222584, RPI.4610, squalamine lactate, SU5416, thalidomide, combretastatin, tamoxifen, COL-3, neovastat, BMS-275291, SU6668, anti-VEGF antibodies, Medi-522 (Vitaxin II), CAI, Interleukin 12, IM862, amiloride, angiostatin, angiostatin K1-3, angiostatin K1-5, captopril, DL-alpha-difluoromethylornithine, DL-alpha-difluoromethylornithine HCl, endostatin, fumagillin, herbimycin A, 4-hydroxyphenylretinamide, juglone, laminin, laminin hexapeptide, laminin pentapeptide, lavendustin A, medroxyprogesterone, minocycline, placental ribonuclease inhibitor, suramin, thrombospondin, antibodies targeted against proangiogenic factors (for example, Avastin, Erbitux, Vectibix, Herceptin); topoisomerase inhibitors; antimicrotubule agents; low molecular weight tyrosine kinases inhibitors of proangiogenic growth factors (for example Tarceva, Nexavar, Sutent, Iressa); GTPase inhibitors; histone deacetylase inhibitors; AKT kinase or ATPase inhibitors; Wnt signaling inhibitors; inhibitors of the E2F transcription factor; mTOR inhibitors (for example Torisel); alpha, beta and gamma interferon, IL-12, matrix metalloproteinase inhibitors (for example, COL3, Marimastat, Batimastat); ZD6474, SU11248, vitaxin; PDGFR inhibitors (for example Gleevec); NM3 and 2-ME2; cyclic peptides such as cilengitide. Other chemotherapy agents suitable are described in detail in The Merck Index in CD-ROM, 13rd Edition.

The methods disclosed in the present invention are useful for predicting the response of a subject suffering from cancer to a chemotherapeutic treatment. The therapy used to treat a cancer depends on the specific kind of cancer. Thus, Table 1 below shows different kinds of cancer and their corresponding chemotherapeutic treatments.

TABLE 1
Cancers and first line corresponding chemotherapeutic treatments

| Types of cancer | Chemotherapeutic treatment |
|---|---|
| Lung cancer | Platinum-based compounds |
| Colon cancer | Antimetabolites |
| Melanoma | Cytokines |
| Pancreatic cancer | Antimetabolites |
| Prostate cancer | Hormonal therapy and for resistant |
| | patients mitotic inhibitors |
| Glioma | DNA-alkylating drugs |
| Bladder cancer | Antimetabolites and platinum |
| | based compounds |
| Ovarian cancer | If epithelial cancer, |
| | platinum-based compounds |
| Hepatobiliary cancer | Antimetabolites or |
| | EGFR-targeted drugs |
| Breast cancer | Hormonal therapy alone, hormonal |
| | therapy combined with cytostatic |
| | cocktails (anthracycline/DNA |
| | alkylating drug/antimetabolite) |
| | or HER2-targeted drugs |
| Lymphoma | CD20-targeted drugs |

The term “platinum-based compound”, as used herein, refers to any compound containing a platinum atom capable of binding and cross-linking DNA, inducing the activation of the DNA repair and ultimately triggering apoptosis. Platinum-based compounds for treating cancer include, without limitation, carboplatin, cisplatin [cis-diamminedichloroplatinum, (CDDP)], oxaliplatin, iproplatin, nedaplatin, triplatin tetranitrate, tetraplatin, satraplatin (JM216), JM118 [cis ammine dichloro (II)], JM149 [cis ammine dichloro (cyclohexylamine) trans dihydroxo platinum (IV)], JM335 [trans ammine dichloro dihydroxo platinum (IV)], transplatin, ZD0473, cis, trans, cis-Pt(NH3)(C6H11NH2)(OOCC3H7) 2C1, malanate-1,2-diaminociclohexanoplatin(II), 5-sulphosalycilate-trans-(1,2-diaminociclohexane)platin (II) (SSP), poly-[(trans-1,2-diaminocyclohexane)platin]-carboxyamilose (POLY-PLAT) and 4-hydroxy-sulphonylphenylacetate (trans-1,2-diaminocyclohexane) platinum (II) (SAP) and the like. In a particular embodiment of the first method of the invention, the platinum-based compound is selected from carboplatin, cisplatin and oxaliplatin; preferably is cisplatin. When the subject suffers from lung cancer or bladder cancer the first line chemotherapeutic treatment is based on platinum-based compounds, preferably cisplatin. When the subject suffers from ovarian cancer, particularly epithelial ovarian cancer, the first line chemotherapeutic treatment is based on platinum-based compounds.

“Antimetabolite”, as used herein, relates, in a broad sense, to substances which disturb normal metabolism and substances which inhibit the electron transfer system to prevent the production of energy-rich intermediates, due to their structural or functional similarities to metabolites that are important for living organisms (such as vitamins, coenzymes, amino acids and saccharides).

Antimetabolites suitable for use in the present invention include, without limitation, folic acid antimetabolites (aminopterin, denopterin, methotrexate, edatrexate, trimetrexate, nolatrexed, lometrexol, pemetrexed, raltitrexed, piritrexim, pteropterin, leucovorin, 10-propargyl-5,8-dideazafolate (PDDF, CB3717)), purine analogs (cladribine, clofarabine, fludarabine, mercaptopurine, pentostatin, thioguanine) and pyrimidine analogs (capecitabine, cytarabine or ara-C, decitabine, fluorouracil, 5-fluorouracil, doxifluridine, floxuridine and gemcitabine). In a preferred embodiment the antimetabolite is selected from 5-fluorouracil and gemcitabine. When the subject suffers from colon cancer the first line chemotherapeutic treatment are antimetabolites, preferably 5-fluorouracil. When the subject suffers from pancreatic cancer, bladder cancer or gallbladder cancer the first line chemotherapeutic treatment are antimetabolites, preferably gemcitabine. When the subject suffers from hepatobiliary cancer, the first line chemotherapeutic treatment is based on antimetabolites, preferably based on fluoropyrimidine. Examples of fluoropyrimidines useful in the treatment of hepatobiliary cancer are 5-fluorouracil, tegafur and capecitabine

The term “cytokines” refers to immunomodulating agents, such as interleukins and interferons, which are polypeptides secreted by specific cells of the immune system and carrying signals locally between cells. Cytokines suitable for use in the present invention are, without limitation, interferon alpha, interferon beta, interferon gamma, interleukin 2, interleukin 12, tumor necrosis factor, granulocyte macrophage colony-stimulating factor (GM-CSF), granulocyte colony-stimulating factor (G-CSF), interleukin 4 (IL-4), interleukin 6 (IL-6), interleukin 18 (IL-18) and interferon alpha 2b. In a preferred embodiment the cytokine used is interferon. When the subject suffers from melanoma the first line chemotherapeutic treatment in stage III are cytokines, preferably interferon.

The term “hormonal therapy” refers to the administration of an anti-tumour agent that acts primarily by interacting with (e.g. interfering with) a hormonal pathway that is specific or relatively specific to particular cell type(s). Said treatment has for purpose to block, inhibit or reduce the effect of hormones, specifically to block the effect of estrogen or progesterone, or alternatively, lower estrogen or progesterone levels, including anti-estrogen or anti-progesterone therapy and estrogen or progesterone ablation therapy. Hormonal therapy includes, without limitation, tamoxifen, toremifene, anastrozole, arzoxifene, lasofoxifene, raloxifene, nafoxidine, fulvestrant, aminoglutethimide, testolactone, atamestane, exemestane, fadrozole, formestane, letrozole, goserelin, leuprorelin or leuprolide, buserelin, histrelin, megestrol and fluoxymesterone. In a preferred embodiment the hormonal therapy is androgen-deprivation therapy. The term “androgen-deprivation therapy” or “androgen suppression therapy” refers to treatments that reduce the levels of the male hormones, androgens, in the body. Androgen-deprivation therapy includes, without limitation, GnRH agonists such as leuprolide, buserelin, goserelin and histrelin. When the subject suffers from prostate cancer, the first line chemotherapeutic treatment is hormonal therapy, preferably androgen-deprivation therapy. When the subject suffers from breast cancer the first line chemotherapeutic treatment is hormonal therapy alone or hormonal therapy combined with cytostatic cocktails. The term “cytostatic cocktail”, in the context of the present invention and related to the treatment of breast cancer, refers to a combination of an anthracycline, a DNA alkylating drug and an antimetabolite. Examples of “cytostatic cocktails”, according to the present invention are, without limitation, FAC (adriamycin/cyclophosphamide/5-fluorouracil), FEC (5-fluorouracil/epirubicin/cyclophosphamide) and CNF (cyclophosphamide/mitoxantrone/5-fluorouracil). In a preferred embodiment the cytostatic cocktail is selected from FAC, FEC and CNF.

The term “mitotic inhibitor” refers to compounds which inhibit mitosis or cell division by disrupting microtubules. Examples of mitotic inhibitors include, without limitation, vinca alkaloids such as vindesine, vincristine, vinblastine, vinorelbine; taxanes such as paclitaxel (Taxol™), docetaxel (Taxotere™); colchicine (NSC 757), thiocolchicine (NSC 361792), colchicine derivatives (e. g., NSC 33410), and allocolchicine (NSC 406042); halichondrin B (NSC 609395); dolastatin 10 (NSC 376128); maytansine (NSC 153858); rhizoxin (NSC 332598); epothilone A, epothilone B; discodermolide; estramustine; nocodazole. In a preferred embodiment the mitotic inhibitor is docetaxel. When the subject suffers from prostate cancer, the second line chemotherapeutic treatment for a cancer that is resistant to hormonal therapy is a treatment with mitotic inhibitors, preferably docetaxel.

“DNA-alkylating drugs”, as used herein, are alkylating agents used in cancer treatment that are capable of adding an alkyl group to DNA of rapidly dividing cells thus leading to replication arrest and cell death. DNA-alkylating agents are nitrogen mustards, nitrosoureas, ethylenimine derivatives, alkyl sulfonates and triazenes, including, but not limited to, cyclophosphamide (Cytoxan™), busulfan, improsulfan, piposulfan, pipobroman, melphalan (L-sarcolysin), chlorambucil, mechlorethamine or mustine, uramustine or uracil mustard, novembichin, phenesterine, trofosfamide, ifosfamide, carmustine (BCNU), lomustine (CCNU), chlorozotocin, fotemustine, nimustine, ranimnustine, semustine (methyl-CCNU), streptozocin, thiotepa, triethylenemelamine, triethylenethiophosphoramine, procarbazine, altretamine, dacarbazine, mitozolomide and temozolomide. In a preferred embodiment the DNA-alkylating drug is selected from temozolomide, nitrosoureas and procarbazine. When the subject suffers from glioma the first line chemotherapeutic treatment are DNA-alkylating drugs, preferably selected from temozolomide, nitrosoureas, procarbazine and combinations thereof.

The term “EGFR-targeted drug”, as used herein, refers to any molecule which is capable of inhibiting totally or partially signaling through EGFR either by targeting the extracellular domain of the receptor and thereby blocking the binding of the ligand to the receptor or by inhibiting the tyrosine kinase activity of the cytoplasmic domain. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225) and reshaped human 225 (H225) (see, WO 96/40210, Imclone Systems Inc.); antibodies that bind type II mutant EGFR (U.S. Pat. No. 5,212,290); humanized and chimeric antibodies that bind EGFR as described in U.S. Pat. No. 5,891,996; and human antibodies that bind EGFR (see WO98/50433, Abgenix), Bevacizumab (Avastin), 2C3, HuMV833, cetuximab (Erbitux®), panitumumab (Vectibix®), nimotuzumab (TheraCim®), matuzumab, zalutuzumab, mAb 806, or IMC-1 1F8. Examples of inhibitors of the tyrosine kinase activity of EGFR include ZD1839 or Gefitinib (IRESSA™; Astra Zeneca), CP-358774 (TARCEVA™; Genentech/OSI) and AG1478, AG1571 (SU 5271; Sugen), erlotinib (Tarceva), sutent (sunitinib), lapatinib, imatinib, sorafenib (nexavar), vandetanib, axitinib, bosutinib, cedivanib, dasatinib (sprycel), lestaurtinib, pazopanib and/or ARQ1 97. In a preferred embodiment the EGFR-targeted drug is sorafenib. When the subject suffers from hepatocelular carcinoma the first line chemotherapeutic treatment is an EGFR-targeted drug, preferably sorafenib.

The term “HER2-targeted drug” refers to a drug directed against the protein human epidermal growth factor receptor 2 (HER2) which is overexpressed in a particular subtype of breast cancers (HER2+). HER2-targeted drugs include, without limitation, trastuzumab, lapatinib, pertuzumab, neratinib, trastuzumab-DM 1 and mTOR inhibitors such as everolimus or temsirolimus. In a preferred embodiment the HER2-targeted drug is trastuzumab. When the subject suffers from breast cancer HER2+ for hormonal receptors, the first line treatment is an HER2-targeted drug, preferably trastuzumab.

The term “CD20-targeted drug” refers to a drug directed to the CD20 antigen on B lymphocytes. CD20-targeted drugs include, without limitation, anti-CD20 antibodies such as rituximab, ocrelizumab, PRO70769, rhuH27, ofatumumab, veltuzumab, hA20, IMMU-106, AME-133, LY2469298, PRO131921, GA-101, tositumomab and RO5072759. In a preferred embodiment the CD20-targeted drug is rituximab. When the subject suffers from a Hodgkin's lymphoma the first line treatment is selected from combined chemotherapy, rituximab and combinations thereof. “Combined chemotherapy” is meant a combination of anticancer drugs that work through different cytotoxic mechanisms. Combined chemotherapy for the treatment of Hodgkin's lymphoma is, without limitation, ABVD (adriamycin/bleomycin/vinblastine/dacarbazine), MOPP (mechlorethamine/vincristine/procarbazine/prednisone), BEACOPP (bleomycin/etoposide/adriamycin/cyclophosphamide/vincristine/procarbazine/prednison e), Stanford V (a mustard derivative such as cyclophosphamide, mechlorethamine or ifosfamide/doxorubicinkinblastine/vincristine/bleomycin/etoposide/prednisone), ChIVPP/EVA (chlorambucil, vincristine, procarbazine, prednisone, etoposide, vinblastine, adriamycin) and VAPEC-B (vincristine/adriamycin/prednisone/etoposide/cyclophosphamide/bleomycin). When the subject suffers from non-Hodgkin's lymphoma the first line chemotherapeutic treatment are combined chemotherapy selected from, without limitation, CHOP (cyclophosphamide/doxorubicin/vincristine/prednisone), CHOP-R or R-CHOP (CHOP+rituximab), COP or CVP (cyclophosphamide/vincristine/prednisone), COPP (cyclophosphamide/vincristine/procarbazine/prednisone), m-BACOD (methotrexate/bleomycin/adriamycin/cyclophosphamide/vincristine/dexamethasone), MACOP-B (methotrexate/leucovorin/adriamycin/cyclophosphamide/vincristine/prednisone/bleomy cin), ProMACE-MOPP (methotrexate/adriamycin/cyclophosphamide/etoposide+MOPP), ProMACE-CytaBOM (prednisone/adriamycin/cyclophosphamide/etoposide/cytarabine/bleomycin/vincristine/methotrexate/leucovorin) and R-FCM (rituximab/fludarabine/cyclophosphamide/mitoxantrone).

Thus, in a preferred embodiment, the predictive method according to the invention further comprises comparing the expression level of ChoKα with a reference value, wherein an alteration in the expression level of the ChoKα gene in said sample with respect to said reference value is indicative of a poor clinical response of the subject to said chemotherapeutic treatment. In yet another embodiment, the alteration in the expression levels of ChoKα is an increase in said expression level with respect to said reference value.

As previously explained, the first method of the invention allows the skilled person to predict the clinical response of a subject suffering from cancer to a chemotherapeutic treatment. In a preferred embodiment the cancer is NSCLC and the chemotherapeutic treatment is a platinum-based chemotherapeutic treatment.

In the context of the present invention, a “platinum-based chemotherapy” or a “platinum-based chemotherapeutic treatment” is understood as any treatment which includes at least a platinum-based compound.

The term “platinum-based compound”, has been defined in detail above and used herein with the same meaning.

As the person skilled in the art understands, in the context of the present invention, a platinum-based chemotherapeutic treatment also includes a combination of a platinum-based compound with one or more chemotherapeutic agents different from a platinum-based compound. Said “chemotherapeutic agent different from a platinum-based compound” may be any agent used in the treatment of NSCLC not included in the aforementioned definition of “platinum-based compound” and includes, without limitation, DNA-alkylating drugs, antimetabolites, mitotic inhibitors, anthracyclines, topoisomerase I and II inhibitors, etc.

The terms “DNA-alkylating drugs”, “antimetabolite” and “mitotic inhibitor” have been described in detail above and are used with the same meaning in the present method.

The term “anthracyclines” refers to antibiotics used in cancer chemotherapy derived from Streptomyces bacteria such as doxorubicin (Adriamycin®), daunorubicin (daunomycin), epirubicin, idarubicin, valrubicin, pirarubicin and mitoxantrone.

“Topoisomerase I and II inhibitors” are agents designed to interfere with the action of topoisomerase enzymes I and II. Topoisomerase I inhibitors include, without limitation, irinotecan, topotecan, camptothecin, acetylcamptothecin, 9-aminocamptothecin, lamellarin D and betulinic acid. Toposomerase II inhibitors include, without limitation, amsacrine, etoposide, teniposide and doxorubicin.

Suitable combinations for the treatment of NSCLC can be, without limitation, cisplatin-paclitaxel, cisplatin-gemcitabine, cisplatin-docetaxel, carboplatin-paclitaxel, cisplatin-etoposide, carboplatin-etoposide, carboplatin-gemcitabine, carboplatin-docetaxel, cisplatin-vinorelbine, carboplatin-vinorelbine, cisplatin-vindesine, cisplatin-teniposide, cisplatin-vindesine, cisplatin-tirapazamine, oxaliplatin-gemcitabine, oxaliplatin-paclitaxel, oxaliplatin-vinorelbine, ZD0473-vinorelbine, ZD0473-paclitaxel, ZD0473-gemcitabine, cisplatin-etoposide-mitomycin C, cisplatin-paclitaxel-gemcitabine, cisplatin-doxorubicin-5-fluorouracil (AFP), cisplatin-cyclophosphamide-bleomycin (CBP), cisplatin-vindesine-mitomycin C (MVP), cyclophosphamide-doxorubicin-cisplatin (CISCA), cisplatin-adriamycin (CA), cisplatin-fluorouracil (CF), cisplatin-gemcitabine-vinorelbine and paclitaxel followed by cisplatin-gemcitabine-vinorelbine.

Therefore, in a particular embodiment, the platinum-based chemotherapeutic treatment is selected from cisplatin-docetaxel, cisplatin-gemcitabine-vinorelbine or paclitaxel followed by cisplatin-gemcitabine-vinorelbine.

The first step of the first method of the invention involves the determination of the expression levels of choline kinase alpha (ChoKα) gene in a sample from the subject under study.

The term “Choline kinase alpha”, as used herein, refers to the alpha isoform of the enzyme which catalyses the phosphorylation of choline in the presence of ATP to produce phosphorylcholine (PCho) (EC 2.7.1.32). Exemplary alpha isoforms of choline kinases the expression of which can be determined according to the present invention include, without limitation, the human ortholog (UniProt accession number P35790), the mouse ortholog (UniProt accession numbers O54804) and the rat ortholog (UniProt accession number Q01134). In a preferred embodiment, the method of the invention comprises the determination of the expression levels of the ChoKα a isoform. In another preferred embodiment, the method of the invention comprises the determination of the expression levels of both ChoKα a and b isoforms. In another preferred embodiment, the method of the invention comprises the determination of the expression levels of the ChoKα b isoform. In a preferred embodiment, the method of the invention comprises the determination of the expression levels of the ChoKα a isoform but does not comprise the determination of the expression levels of the b isoform. In another preferred embodiment, the method of the invention comprises the determination of the expression levels of the ChoKα b isoform but does not comprise the determination of the expression levels of the ChoKα a isoform.

The term “ChoKα a isoform” “ChoKα isoform 1” or “ChoKα long isoform” are used herein interchangeably to refer to a polypeptide of 457 amino acids which is provided in the NCBI database under accession number NP_{—}001268 (release of Jun. 17, 2012). The polypeptide is encoded by a 2733 bp transcript which is formed by alternative splicing from the CHKA gene. The cDNA sequence of the transcript encoding the a isoform is provided in the NCBI database with accession number NM_{—}001277 (release of Jun. 17, 2012).

The term “ChoKα b isoform”, “ChoKα isoform 2” or “ChoKα short isoform”, are used herein interchangeably to refer to a polypeptide of 439 amino acids which is provided in the NCBI database under accession number NP_{—}997634 (release of Jun. 17, 2012). The polypeptide is encoded by a 2679 bp transcripts which is formed by alternative splicing from the CHKA gene. The cDNA sequence of the transcript encoding the b isoform is provided in the NCBI database with accession number NM_NM_{—}212469 (release of Jun. 17, 2012).

The term “sample”, as used herein, relates to any sample which can be obtained from the subject. The present method can be applied to any kind of biological sample from a subject, such as a biopsy sample, tissue, cell or fluid (serum, saliva, semen, sputum, cerebral spinal fluid (CSF), tears, mucus, sweat, milk), brain extracts, samples obtained by bronchial lavage, bronchoscopy, fine needle aspiration biopsy (FNAB) and the like. In a particular embodiment, said sample is a tissue sample, preferably a tumour tissue sample, more preferably a lung tumour tissue sample from a subject suffering from cancer, preferably from a subject suffering from NSCLC. Said sample can be obtained by conventional methods, e.g., biopsy, by using methods well known to those of ordinary skill in the related medical arts. Methods for obtaining the sample from the biopsy include gross apportioning of a mass, or microdissection or other art-known cell-separation methods. Tumour cells can additionally be obtained from fine needle aspiration cytology. In a preferred embodiment samples are obtained by bronchial lavage. In another preferred embodiment samples are obtained by fine needle aspiration biopsy (FNAB). In order to simplify conservation and handling of the samples, these can be formalin-fixed and paraffin-embedded or first frozen and then embedded in a cryosolidifiable medium, such as OCT-Compound, through immersion in a highly cryogenic medium that allows for rapid freeze (OCT embedded frozen tissue).

In a particular embodiment of the present invention, the expression levels of the ChoKα gene can be determined by measuring the levels of mRNA encoded by said gene, or by measuring the levels of the protein encoded by said gene, i.e. ChoKα protein, or of variants thereof.

In order to measure the mRNA levels of ChoKα gene, the biological sample may be treated to physically, mechanically or chemically disrupt tissue or cell structure, to release intracellular components into an aqueous or organic solution to prepare nucleic acids for further analysis. The nucleic acids are extracted from the sample by procedures known to the skilled person and commercially available. RNA is then extracted from frozen or fresh samples by any of the methods typical in the art, for example, Sambrook, J., et al., 2001. Molecular cloning: A Laboratory Manual, 3^{rd} ed., Cold Spring Harbor Laboratory Press, N.Y., Vol. 1-3. Preferably, care is taken to avoid degradation of the RNA during the extraction process.

The expression level can be determined using mRNA obtained from a formalin-fixed, paraffin-embedded tissue sample. mRNA may be isolated from an archival pathological sample or biopsy sample which is first deparaffinized. An exemplary deparaffinization method involves washing the paraffinized sample with an organic solvent, such as xylene. Deparaffinized samples can be rehydrated with an aqueous solution of a lower alcohol. Suitable lower alcohols, for example, include methanol, ethanol, propanols and butanols. Deparaffinized samples may be rehydrated with successive washes with lower alcoholic solutions of decreasing concentration, for example. Alternatively, the sample is simultaneously deparaffinized and rehydrated. The sample is then lysed and RNA is extracted from the sample. Samples can be also obtained from fresh tumour tissue.

In a preferred embodiment samples can be obtained from fresh tumour tissue or from OCT embedded frozen tissue. In another preferred embodiment samples can be obtained by bronchoscopy and then paraffin-embedded.

Determination of the levels of ChoKα mRNA can be carried out by any method known in the art such as qPCR, northern blot, RNA dot blot, TaqMan, tag based methods such as serial analysis of gene expression (SAGE) including variants such as LongSAGE and SuperSAGE, microarrays. Determination of the levels of the ChoKα mRNA can also be carried out by Fluorescence In Situ Hybridization, including variants such as Flow-FISH, qFiSH and double fusion fish (D-FISH) as described in WO2010030818, Femino et al. (Science, 1998, 280:585-590), Levsky et al. (Science, 2002, 297:836-840) or Raj et al. (PLoS Biology, 2006, 4:e309). The levels of ChoKα mRNA can also be determined by nucleic acid sequence based amplification (NASBA) technology.

In a preferred embodiment, the gene mRNA expression levels are often determined by reverse transcription polymerase chain reaction (RT-PCR). The detection can be carried out in individual samples or in tissue microarrays.

Thus, in a particular embodiment, the mRNA expression levels of ChoKα gene are determined by quantitative PCR, preferably, Real-Time PCR. The detection can be carried out in individual samples or in tissue microarrays.

In order to normalize the values of mRNA expression among the different samples, it is possible to compare the expression levels of the mRNA of interest in the test samples with the expression of a control RNA. A “control RNA” as used herein, relates to RNA whose expression levels do not change or change only in limited amounts in tumour cells with respect to non-tumorigenic cells. Preferably, the control RNA is mRNA derived from housekeeping genes and which code for proteins which are constitutively expressed and carry out essential cellular functions. Preferred housekeeping genes for use in the present invention include β-2-microglobulin, ubiquitin, 18-S ribosomal protein, cyclophilin, GAPDH, PSMB4, tubulin and β-actin. In a preferred embodiment, the control RNA is GAPDH, β-actin, 18-S ribosomal protein or PSMB4 mRNA.

In one embodiment relative gene expression quantification is calculated according to the comparative Ct method using GAPDH, β-actin or PSMB4 as an endogenous control and commercial RNA controls as calibrators. Final results are determined according to the formula 2-(ΔCt sample-ΔCt calibrator), where ΔCT values of the calibrator and sample are determined by subtracting the CT value of the target gene from the value of the control gene.

Alternatively, in another embodiment of the first method of the invention, the expression levels of ChoKα gene are determined by measuring the expression of the ChoKα protein or of variants thereof. In a preferred embodiment the expression levels of ChoKα protein or of variants thereof are determined by Western blot or by immunohistochemistry.

The expression levels of ChoKα protein can be quantified by means of conventional methods, for example, using antibodies with a capacity to specifically bind to ChoKα protein (or to fragments thereof containing antigenic determinants) and subsequent quantification of the resulting antibody-antigen complexes.

The antibodies to be employed in these assays can be, for example, polyclonal sera, hybridoma supernatants or monoclonal antibodies, antibody fragments, Fv, Fab, Fab′ and F(ab′)2, ScFv, diabodies, triabodies, tetrabodies and humanised antibodies. At the same time, the antibodies can be labelled or not. Illustrative, but non-exclusive examples of markers which can be used include radioactive isotopes, enzymes, fluorophores, chemiluminescent reagents, enzymatic substrates or cofactors, enzymatic inhibitors, particles, colorants, etc. There are a wide variety of well-known assays that can be used in the present invention, which use non-labelled antibodies (primary antibody) and labelled antibodies (secondary antibodies); among these techniques are included Western blot or Western transfer, ELISA (enzyme linked immunosorbent assay), RIA (radioimmunoassay), competitive EIA (enzymatic immunoassay), DAS-ELISA (double antibody sandwich ELISA), immunocytochemical and immunohistochemical techniques, techniques based on the use of biochips or protein microarrays including specific antibodies or assays based on colloidal precipitation in formats such as dipsticks. Other ways of detecting and quantifying the ChoKα protein include techniques of affinity chromatography, binding-ligand assays, etc.

On the other hand, the determination of ChoKα protein expression levels can be carried out by constructing a tissue microarray (TMA) containing the subject samples assembled, and determining the expression levels of ChoKα protein by immunohistochemistry techniques Immunostaining intensity can be evaluated by two different pathologists and scored using uniform and clear cut-off criteria, in order to maintain the reproducibility of the method. Discrepancies can be resolved by simultaneous re-evaluation. Briefly, the result of immunostaining can be recorded as negative expression (0) versus positive expression, and low expression (1+) versus moderate (2+) and high (3+) expression, taking into account the expression in tumour cells and the specific cut-off for each marker. As a general criterion, the cut-offs were selected in order to facilitate reproducibility, and when possible, to translate biological events. Alternatively, the immunostaining intensity can be evaluated by using imaging techniques and automated methods such as those disclosed in Rojo, M. G. et al. (Folia Histochem. Cytobiol. 2009; 47(3): 349-54) or Mulrane, L. et al. (Expert Rev. Mol. Diagn. 2008; 8(6):707-25).

Alternatively, in another particular embodiment, the expression levels of ChoKα protein or of variants thereof are determined by Western blot. Western blot is based on the detection of proteins previously resolved by gel electrophoreses under denaturing conditions and immobilized on a membrane, generally nitrocellulose, by the incubation with an antibody specific and a developing system (e.g. chemoluminiscent).

As previously mentioned, variants of the ChoKα protein can be used for measuring the expression levels of the ChoKα gene in order to put into practice the first method of the invention.

Human ChoKα gene encodes two isoforms of ChoKα protein produced by alternative splicing. Isoform 1 has 457 amino acids, and isoform 2 has 439 amino acids because positions 155-172 are missing. Moreover, some natural variants have been described.

Thus, variants of the ChoKα protein may be: (i) one in which one or more of the amino acid residues are substituted with a conserved or non-conserved amino acid residue (preferably a conserved amino acid residue) and such substituted amino acid residue may or may not be one encoded by the genetic code; (ii) one in which there are one or more modified amino acid residues, e.g., residues that are modified by the attachment of substituent groups; (iii) one in which the protein is an alternative splice variant of the proteins of the present invention and/or; (iv) fragments of the proteins. The fragments include proteins generated via proteolytic cleavage (including multi-site proteolysis) of an original sequence. Variants are deemed to be within the scope of those skilled in the art from the teaching herein.

Variants according to the present invention include amino acid sequences that are at least 60%, 70%, 80%, 90%, 95% or 96% similar or identical to the original amino acid sequence. As known in the art the “similarity” between two proteins is determined by comparing the amino acid sequence and its conserved amino acid substitutes of one protein to a sequence of a second protein. The degree of identity between two proteins is determined using computer algorithms and methods that are widely known for the persons skilled in the art. The identity between two amino acid sequences is preferably determined by using the BLASTP algorithm [BLASTManual, Altschul, S., et al., NCBI NLM NIH Bethesda, Md. 20894, Altschul, S., et al., J. Mol. Biol. 215: 403-410 (1990)].

The proteins can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Additionally, the proteins may include unnatural amino acids formed by post-translational modification or by introducing unnatural amino acids during translation.

In a particular embodiment said variant is a mammal variant, preferably a human variant, more preferably with at least 60%, 70%, 80%, 90%, 95% or 96% similarity or identity to the original amino acid sequence.

In a preferred embodiment, the first method of the invention further comprises comparing the expression levels of ChoKα with reference values, wherein an alteration in the expression levels of ChoKα gene in said sample with respect to said reference values are indicative of a poor clinical response of the subject to said chemotherapeutic treatment or of a good clinical response of the subject to said chemotherapeutic treatment.

In a preferred embodiment, once the expression levels of choline kinase alpha (ChoKα) gene have been determined in a sample, the first method of the invention further comprises comparing said expression levels with a reference value wherein an alteration in the expression level of ChoKα gene in said sample with respect to said reference value is indicative of a poor clinical response of the subject to said chemotherapeutic treatment or of a good clinical response of the subject to said chemotherapeutic treatment.

The reference value can be determined by techniques well known in the state of the art, for example, determining the median value of expression levels of ChoKα gene measured in a collection of tumour tissue in biopsy samples from subjects suffering from cancer who have or not received a chemotherapeutic treatment, or from normal tissue. In a preferred embodiment the expression levels of ChoKα gene are measured in a collection of tumour tissue in biopsy samples from subjects suffering from NSCLC who have or not received a platinum-based chemotherapeutic treatment, or from normal lung tissue. Once this median value is established, the level of this marker expressed in tumour tissues from the subject can be compared with this median value, and thus be assigned a level of “decreased” (low) or “increased” (high) expression level. The collection of samples from which the reference level is derived will preferably be constituted from subjects suffering from the same type of cancer, i.e. NSCLC, or a mixture of lung tissues from normal individuals not affected of lung cancer. Alternatively, the use of a reference value used for determining whether the expression level of a gene is “increased” or “decreased” could correspond to the median value of expression levels of ChoKα gene measured in a RNA sample obtained by pooling equal amounts of RNA from each of the tumour samples obtained by biopsy from subjects suffering from cancer who have or not received a chemotherapeutic treatment, preferably from subjects suffering from NSCLC who have or not received a platinum-based chemotherapeutic treatment. Once this median value is established, the level of this marker expressed in tumour tissues from subjects can be compared with this median value, and thus be assigned a level of “increased”, “decreased” or “lack of change”. For example, an increase in expression levels above the reference value of at least 1.1-fold, 1.5-fold, 5-fold, 10-fold, 20-fold, 30-fold, 40-fold, 50-fold, 60-fold, 70-fold, 80-fold, 90-fold, 100-fold or even more compared with the reference value is considered as “increased” expression level. On the other hand, a decrease in expression levels below the reference value of at least 0.9-fold, 0.75-fold, 0.2-fold, 0.1-fold, 0.05-fold, 0.025-fold, 0.02-fold, 0.01-fold, 0.005-fold or even less compared with reference value is considered as “decreased” expression level. A “lack of change” in the expression levels with respect to a reference value refers to expression levels which are substantially unaltered with respect to the reference value. For instance, a lack of change in the expression in the sample under study is considered when the levels differ by no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5%, no more than 6%, no more than 7%, no more than 8%, no more than 9%, no more than 10% or no more than the percentage value that is the same as the error associated to the experimental method used in the determination.

An increased or decreased expression level of ChoKα gene is considered an alteration in the expression levels of ChoKα gene. In a preferred embodiment of the first method of the invention the alteration in the expression levels of ChoKα is an increase in said expression level with respect to said reference value. In another embodiment of the first method of the invention the alteration in the expression levels of ChoKα is a decrease in said expression level with respect to said reference value

In the present invention, the “reference value” is an arbitrary cut-off point, established according to ROC methodology. Once this cut-off point is established, the level of this marker expressed in tumour tissues from the subject can be compared with this cut-off point, and thus be assigned a level of “low” expression if it is under this cut-off, or a level of “high” expression when it is above this cut-off.

Once a comparison has been made between the expression levels of the ChoKα gene and the reference value, the method of the invention allows making a prediction as to whether the subject will show a poor or a good clinical response to the chemotherapeutic treatment, preferably to the platinum-based chemotherapeutic treatment. In particular, the increase in said expression level is indicative of a poor clinical response or the decrease in said expression level is indicative of a good clinical response.

The terms “poor” or “good”, as used herein to refer to a clinical response, refer that the subject will show a favourable or unfavourable response to the chemotherapy, preferably to the platinum-based chemotherapy. As will be understood by those skilled in the art, such the assessment of the probability, although preferred to be, may usually not be correct for 100% of the subjects to be diagnosed. The term, however, requires that a statistically significant portion of subjects can be identified as having a predisposition therefore or of not responding to the chemotherapeutic treatment, preferably to a platinum-based chemotherapeutic treatment. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% at least 95%. The p-values are, preferably, 0.1, 0.05, 0.01, 0.005, or 0.0001. More preferably, at least 60 percent, at least 70 percent, at least 80 percent or at least 90 percent of the subjects of a population can be properly identified by the method of the present invention.

### Methods for Designing an Individualised Therapy for a Cancer Patient

The findings of the inventors can also be used for designing an individual therapy for a subject suffering from cancer, preferably NSCLC, based on the expression levels of ChoKα gene. As shown in the experimental part of the present invention, subjects suffering from NSCLC having high expression levels of ChoKα gene are less likely to respond to a platinum-based chemotherapeutic treatment. Thus, these subjects are candidates for first line treatment with therapies generally used in second line in subjects not responding to platinum-based chemotherapy. In this way, subjects can proceed directly to adequate therapies while avoiding the secondary effects associated with platinum-based therapy.

Thus, in another aspect, the invention relates to an in vitro method (hereinafter second method of the invention) for designing an individual therapy for a subject suffering from cancer comprising determining the expression levels of choline kinase alpha (ChoKα) gene in a sample from the subject. In a preferred embodiment, the method of the invention comprises the determination of the expression levels of the ChoKα a isoform. In another preferred embodiment, the method of the invention comprises the determination of the expression levels of both ChoKα a and b isoforms. In another preferred embodiment, the method of the invention comprises the determination of the expression levels of the ChoKα b isoform. In a preferred embodiment, the method of the invention comprises the determination of the expression levels of the ChoKα a isoform but does not comprise the determination of the expression levels of the b isoform. In another preferred embodiment, the method of the invention comprises the determination of the expression levels of the ChoKα b isoform but does not comprise the determination of the expression levels of the a isoform.

The terms “subject”, “cancer”, “choline kinase alpha” and “subject” have been described in detail above in the context of the first method of the invention and are used with the same meaning in the context of the second method of the invention.

In a preferred embodiment the second method of the invention further comprises comparing the expression levels of ChoKα with a reference value,
wherein a decrease or a lack of change in the expression level of ChoKα gene in said sample with respect to said reference value is indicative that the subject is a candidate for a therapy based on said chemotherapeutic treatment
or
wherein an increase in the expression level of ChoKα gene in said sample with respect to said reference value is indicative that the subject is a candidate for the treatment with a therapy selected from the group consisting of:
(i) a ChoKα inhibitor,
(ii) a folate antimetabolite,
(iii) an antimicrotubule agent,
(iv) an EGFR-targeted drug,
(v) a combination of one or more of (i) to (iv) above.

In a preferred embodiment of the second method of the invention the cancer is NSCLC.

In yet another embodiment, the chemotherapeutic treatment is a platinum-based chemotherapeutic treatment. The terms “NSCLC”, “chemotherapy” and “platinum-based chemotherapeutic treatment” have been described in detail in the context of the predictive method of the invention and are used with the same meaning in the context of the second method of the invention.

In another embodiment, those subjects showing high expression levels of choline kinase alpha (ChoKα) gene are candidates for the treatment with other therapies used as second line in non-responders such as:

(i) a ChoKα inhibitor,

(ii) a folate antimetabolite,

(iii) an antimicrotubule agent,

(iv) an EGFR-targeted drug,

(v) a combination of one or more of (i) to (iii) above

The term “ChoKα inhibitor”, as used herein, is understood as any compound capable of producing a decrease in the ChoKα activity, including those compounds which prevent the expression of the ChoKα gene, causing reduced levels of mRNA or ChoKα protein, as well as compounds which inhibit ChoKα causing a decrease in the activity of the enzyme.

Compounds capable of preventing the expression of the ChoKα gene can be identified using standard assays for determining the mRNA expression levels such as RT-PCR, RNA protection analysis, Northern procedure, in situ hybridization, microarray technology and the like.

The compounds which cause reduced levels of ChoKα protein can be identified using standard assays for determining the protein expression levels such as immunoblot or Western blot, ELISA (adsorption enzyme immunoanalysis), RIA (radioimmunoassay), competitive EIA (competitive enzyme immunoassay), DAS-ELISA (double antibody sandwich ELISA), immunocytochemical and immunohistochemical techniques, techniques based on the use of protein microarrays or biochip which include specific antibodies or assays based on colloidal precipitation in formats such as reagent strips.

The determination of the inhibiting capacity on the biological activity of choline kinase is detected using standard assays to measure the activity of choline kinase, such as methods based on the detection of the phosphorylation of choline labeled with [14C] by ATP in the presence of purified recombinant choline kinase or a choline kinase-rich fraction followed by detection of the phosphorylated choline using standard analytical techniques (for example, TLC) as described in EP1710236.

Exemplary choline kinase alpha inhibitors that can be used in non-responders to platinum-based chemotherapy are described in Table 2 from I to XVII.

TABLE 2
ChoKα inhibitors

| I | Compounds as described in U.S. patent application US20070185170 | | | |
|---|---|---|---|---|
| | (granted as U.S. Pat. No. 7,781,458) having general formula | | | |
| | 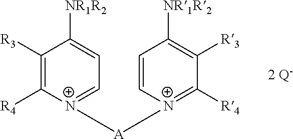 | | | |
| | wherein | | | |
| | Q⁻ represents the conjugate base of a pharmaceutically suitable | | | |
| | organic or inorganic acid; | | | |
| | R₁ and R′₁ represent, independently of each other, an aryl radical | | | |
| | optionally substituted with halogen, trifluoromethyl, hydroxyl, | | | |
| | C₁₋₆ alkyl, amino or alkoxyl; | | | |
| | R₂ and R′₂ represent, independently of each other, an aryl radical | | | |
| | optionally substituted with halogen, trifluoromethyl, hydroxyl, | | | |
| | C₁₋₆ alkyl, amino or alkoxyl; | | | |
| | R₃ and R′₃ represent, independently of each other, either a | | | |
| | radical selected from the group consisting of H, halogen, | | | |
| | trifluoromethyl, hydroxyl, amino, alkoxyl and C₁₋₆ alkyl | | | |
| | optionally substituted with trifluoromethyl, hydroxyl, amino or | | | |
| | alkoxyl, or together with R₄ and R′₄, respectively, and | | | |
| | independently of each other, a —CH═CH—CH═CH— radical | | | |
| | optionally substituted with halogen, trifluoromethyl, hydroxyl, | | | |
| | C₁₋₆ alkyl, amino or alkoxyl; | | | |
| | R₄ and R′₄ represent, independently of each other, either a | | | |
| | radical selected from the group consisting of H and C₁₋₆ alkyl | | | |
| | optionally substituted with halogen, trifluoromethyl, hydroxyl, | | | |
| | amino or alkoxyl, or together with R₃ and R′3, respectively, and | | | |
| | independently of each other, a —CH═CH—CH═CH— radical | | | |
| | optionally substituted with halogen, trifluoromethyl, hydroxyl, | | | |
| | C₁₋₆ alkyl, amino or alkoxyl; | | | |
| | A represents a spacer group comprising any divalent organic | | | |
| | structure acting as a bond between the two pyridinium groups | | | |
| | present in the structure defined by means of formula I and, | | | |
| | particularly, divalent molecules having a structure selected from | | | |
| | the group of: | | | |
| | 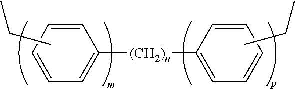 | | | |
| | where m, n and p represent integers which can have the | | | |
| | following values: m = 0, 1; n = 0, 1-10; p = 0, 1; on the condition | | | |
| | that m, n and p do not take the value of zero at the same time. | | | |
| | 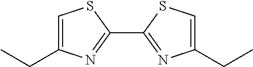 | | | |
| | 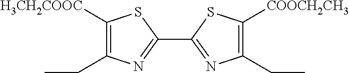 | | | |
| | 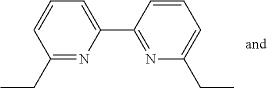 [SMILES: CCc1cccc(-c2cccc(CC)n2)n1] | | | |
| | 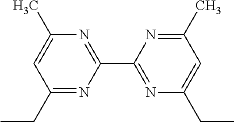 [SMILES: CCc1cc(C)nc(-c2nc(C)cc(CC)n2)n1] | | | |
| | The preferred compounds in this group include those in which | | | |
| | the substituents NR₁R₂, R₃, R₄ and A are as follows: | | | |
| | Compound | R₃, R₄ | NR₁R₂ | A |
| | 1 | H, H | 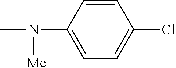 | 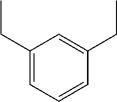 |
| | 2 | H, H | 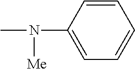 | 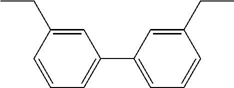 [SMILES: CCc1cccc(-c2cccc(CC)c2)c1] |
| | 3 | H, H | 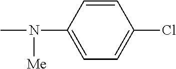 | 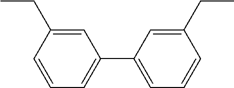 [SMILES: CCc1cccc(-c2cccc(CC)c2)c1] |
| | 4 | H, H | 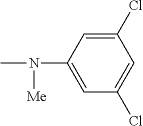 | 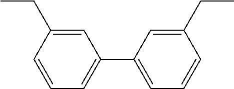 [SMILES: CCc1cccc(-c2cccc(CC)c2)c1] |
| | 5 | —(CH═CH)₂— | 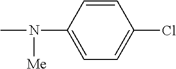 | 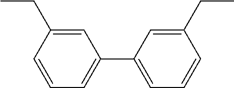 [SMILES: CCc1cccc(-c2cccc(CC)c2)c1] |
| | 6 | —C⁵H═C⁶H—C⁷Cl═C⁸H— | 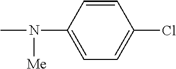 | 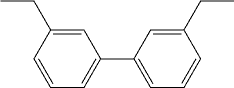 [SMILES: CCc1cccc(-c2cccc(CC)c2)c1] |
| | 7 RSM932-A | —(CH═CH)₂— | 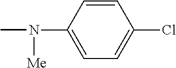 | 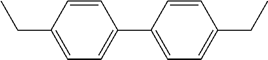 [SMILES: CCc1ccc(-c2ccc(CC)cc2)cc1] |
| | 8 | —C⁵H═C⁶H—C⁷Cl═C⁸H— | 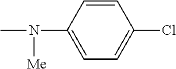 | 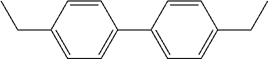 [SMILES: CCc1ccc(-c2ccc(CC)cc2)cc1] |
| | 9 | —(CH═CH)₂— | 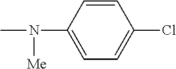 | 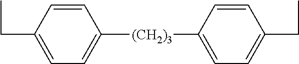 |
| | 10 | —C⁵H═C⁶H—C⁷Cl═C⁸H— | 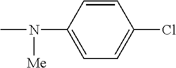 | 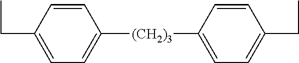 |

| | The preferred compounds in this group include 4-(4-chloro-N- | |
|---|---|---|
| | methylaniline)quinoline and 7-chloro-4-(4-chloro-N- | |
| | methylamino)quinoline having the structures | |
| | 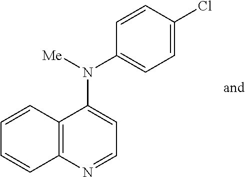 [SMILES: CN(c1ccc(Cl)cc1)c1ccnc2ccccc12] | |
| | 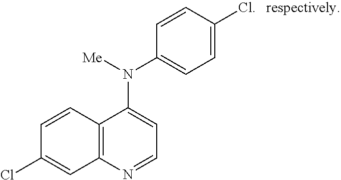 [SMILES: CN(c1ccc(Cl)cc1)c1ccnc2cc(Cl)ccc12] | |
| II | Compounds as described in international patent application WO9805644 | |
| | having the general structural formula | |
| | 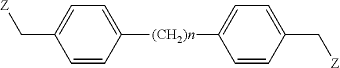 | |
| | wherein | |
| | n is 0, 1, 2 or 3 | |
| | Z is any structural group selected from the group of | |
| | 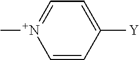 | A |
| | 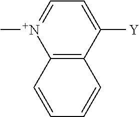 | B |
| | 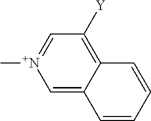 | C |
| | 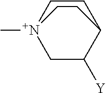 | D |

| wherein Y is selected from the group of —H, —CH₃, —CH₂—OH, | |
|---|---|
| —CO—CH₃, —CN, —NH₂, —N(CH₃)₂, pyrrolidine, piperidine, | |
| perhydroazepine, —OH, —O—CO—C₁₅H₃₁, etc. | |
| The preferred ChoK inhibitors having the formula defined above | |
| are compounds 1 to 6 described by Conejo-García et al. | |
| (J. Med. Chem., 2003, 46: 3754-3757) having the following | |
| structures | |
| 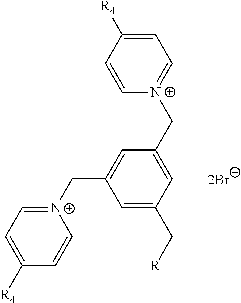 | 2 |

| wherein R is H or | |
|---|---|
| 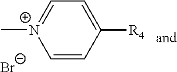 | |
| 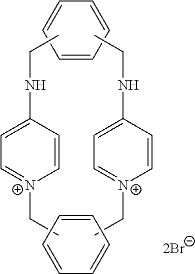 | |
| Compound | isomer |
| 3 | p, p |
| 4 | m, m |
| 5 | p, m |
| 6 | m, p |

| | The compounds which are in the previous general formula are | |
|---|---|---|
| | selected from the group of GRQF-JCR795b, GRQF-MN94b and | |
| | GRQF-MN58b having the structures | |
| | 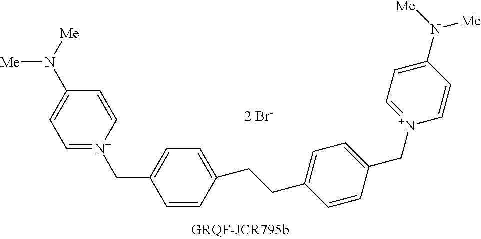 [SMILES: CN(C)c1cc[n+](Cc2ccc(CCc3ccc(C[n+]4ccc(N(C)C)cc4)cc3)cc2)cc1] | |
| | 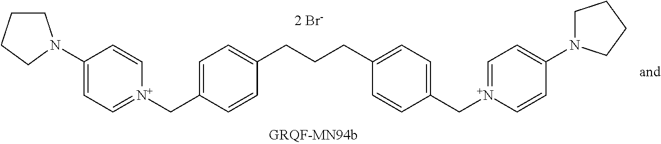 [SMILES: c1cc(C[n+]2ccc(N3CCCC3)cc2)ccc1CCCc1ccc(C[n+]2ccc(N3CCCC3)cc2)cc1] | |
| | 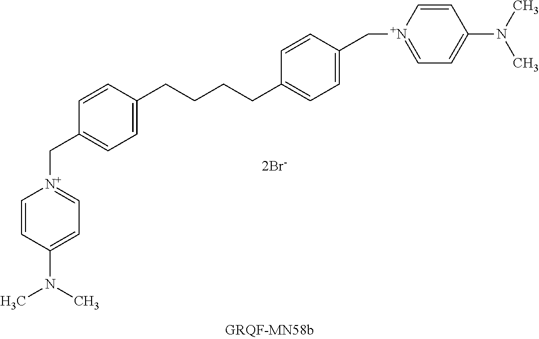 [SMILES: CN(C)c1cc[n+](Cc2ccc(CCCCc3ccc(C[n+]4ccc(N(C)C)cc4)cc3)cc2)cc1] | |
| III | Compounds as described in international patent application WO9805644 | |
| | having the general structural formula | |
| | 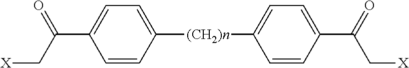 | |
| | wherein | |
| | n is 0, 1, 2, 3, etc. | |
| | X is a structural element selected from the group of A, B, C, D and | |
| | E as follows | |
| | 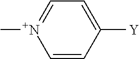 | A |
| | 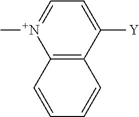 | B |
| | 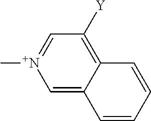 | C |
| | 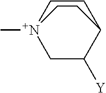 | D |
| | 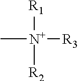 | E |
| | wherein Y is selected from —H, —CH₃, —CH₂—OH, —CO—CH₃, —CN, | |
| | —NH₂, —N(CH₃)₂, pyrrolidine, piperidine, perhydroazepine, —OH, | |
| | —O—CO—C₁₅H₃₁ | |
| | and wherein R₁, R₂ and R₃ are alkyl groups such as —Me and —Et | |
| | and the like although in some cases, R₂ and R₃ can be more | |
| | complex groups such as —CH₂—CH(OMe)₂ and —CH₂—CH(OEt)₂. | |
| | The preferred compounds having the previous general structure | |
| | are GRQF-FK3 and GRQF-FK21 having the following structures: | |
| | 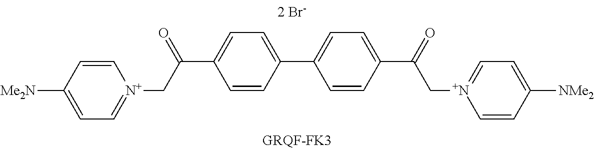 [SMILES: CN(C)c1cc[n+](CC(=O)c2ccc(-c3ccc(C(=O)C[n+]4ccc(N(C)C)cc4)cc3)cc2)cc1] | |
| | 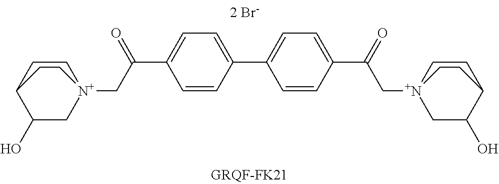 [SMILES: O=C(C[N+]12CCC(CC1)C(O)C2)c1ccc(-c2ccc(C(=O)C[N+]34CCC(CC3)C(O)C4)cc2)cc1] | |
| IV | Compounds as described in international patent application WO9805644 | |
| | having the general structural formula | |
| | 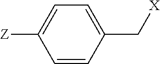 | |
| | wherein X is a group selected from the group of A, B, C and D as | |
| | follows | |
| | 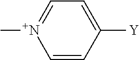 | A |
| | 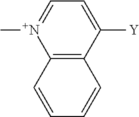 | B |
| | 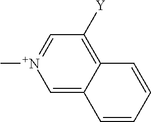 | C |
| | 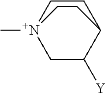 | D |
| | wherein Y is a substituent such as —H, —CH₃, —CH₂OH, —CN, | |
| | —NH2, —N(CH₃)₂, pyrrolidinyl, piperidinyl, perhydroazepine, —OH, | |
| | —O—CO—C₁₅H₃₁ and the like | |
| | wherein Z is an alkyl (—Me, —Et, etc.), aryl, phenyl group, or | |
| | electron donor groups such as —OMe, —NH₂, —NMe₂, etc. | |
| | The preferred compounds having the previous general structure | |
| | are GRQF-MN98b and GRQF-MN164b having the following | |
| | structures: | |
| | 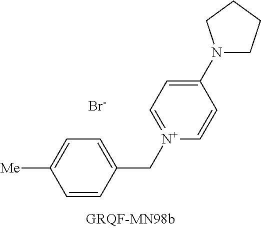 [SMILES: Cc1ccc(C[n+]2ccc(N3CCCC3)cc2)cc1 ; [Br-]] | |
| | 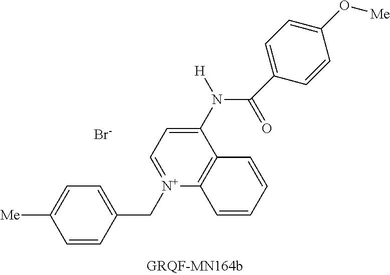 [SMILES: COc1ccc(C(=O)Nc2cc[n+](Cc3ccc(C)cc3)c3ccccc23)cc1 ; [Br-]] | |
| V | Compounds as described in international patent application WO9805644 | |
| | having the general structural formula | |
| | 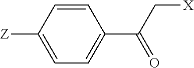 | |
| | wherein X is a group selected from the group of A, B, C and D as | |
| | follows | |
| | 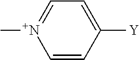 | A |
| | 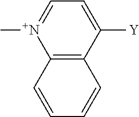 | B |
| | 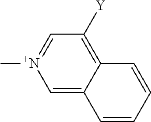 | C |
| | 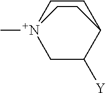 | D |

| | wherein Y is a substituent such as —H, —CH₃, —CH₂OH, | |
|---|---|---|
| | —CO—CH₃, —CN, —NH₂, —N(CH₃)₂ | |
| | wherein Z is an alkyl (—Me, —Et, etc.), aryl (phenyl and the like) | |
| | group, or electron donor groups such as —OMe, —NH₂, —NMe₂, etc. | |
| | The preferred compounds having the previously mentioned | |
| | structure are GRQF-FK29 and GRQF-FK33 having the following | |
| | structures | |
| | 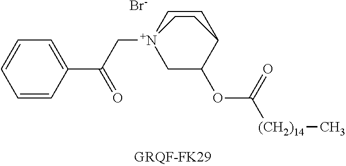 | |
| | 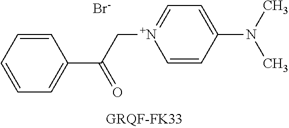 [SMILES: CN(C)c1cc[n+](CC(=O)c2ccccc2)cc1 ; [Br-]] | |
| VI | Compounds described in international patent application WO2004016622 | |
| | having the general structural formula | |
| | 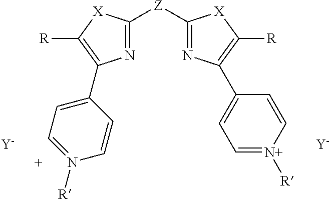 | |
| | wherein X is oxygen or sulfur, | |
| | Z is a single bond, 1,2-ethylidene, isopropylidene, p,p′-biphenyl, | |
| | p-phenyl, m-phenyl, 2,6-pyridylene, p,p′-oxydiphenyl or p,p ′- | |
| | hexafluoroisopropylidene diphenyl; | |
| | R is H, alkyl, alkyldiene, alkyne, aryl, halogen, alcohol, thiol, | |
| | ether, thioether, sulfoxides, sulfones, substituted or primary | |
| | amines, nitro, aldehydes, ketones, nitrile, carboxylic acids, | |
| | derivatives and sulfates thereof, methanesulfonate, hydrochloride, | |
| | phosphate, nitrate, acetate, propionate, butyrate, palmitate, oxalate, | |
| | malonate, maleate, malate, fumarate, citrate, benzoate, | |
| | R′ is H or alkyl | |
| | Y is H or sulfate, methanesulfonate, hydrochloride, phosphate, | |
| | nitrate, acetate, propionate, butyrate, palmitate, oxalate, malonate, | |
| | maleate, malate, fumarate, citrate or benzoate. | |
| | In a preferred embodiment, the compounds having the previously | |
| | defined structure are selected from the group of 2,2-bis[(5-methyl- | |
| | 4-(4-pyridyl)-2-oxazolyl)]propane, 2,2-bis[(5-trifluoromethyl-4- | |
| | (4-pyridyl)-2-oxazolyl)]propane, 4,4′-bis[(5-trifluoromethyl-4-(1- | |
| | methyl-4-pyridinium)-2-oxazolyl)]biphenyl, 4,4′-bis[(5- | |
| | pentafluoroethyl-4-(1-methyl-4-pyridinium)-2-oxazolyl)]biphenyl, | |
| | 4,4′-bis[(5-trifluoromethyl-4-(1-methyl-4-pyridinium)-2- | |
| | oxazolyl)]hexafluoroisopropylidenediphenyl, 2,2-bis[(5- | |
| | trifluoromethyl-4-(4-pyridyl)-2-thiazolyl)]propane and 4,4′-bis[(5- | |
| | trifluoromethyl-4-(1-methyl-4-pyridinium)-2-thiazolyl)]-1,1′- | |
| | oxybisbenzene. | |
| VII | Hemicholinium-3 described in Cuadrado et al. (Oncogene, 1993, 8: 2959- | |
| | 2968) and Jiménez et al. (J. Cell Biochem., 57: 141-149) and Hernández- | |
| | Alcoceba, et al. (Oncogene, 1997, 15: 2289-2301). | |
| VIII | A compound as defined in international patent application WO2007077203 | |
| | having a general structure of the formula | |
| | 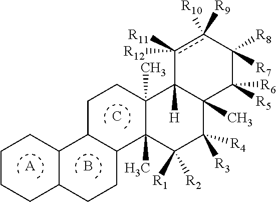 | |
| | wherein | |
| | R₁, R₂, R₃, R₄, R₅, R₆, R₇, R₈, R₁₁ and R₁₂ are independently | |
| | hydrogen; hydroxyl; halogen; substituted or non-substituted C₁₋C₁₂ | |
| | alkyl; substituted or non-substituted C₆₋C₁₀ aryl; a N(R′)(R″) | |
| | amino group, where R′ and R″ are independently hydrogen or a | |
| | C₁₋C₁₂ alkyl group; an OCOR group, where R is (CH₂)₂—COOH or | |
| | (CH₂)₂CO₂CH₂CH₃; or each pair can form a (C═O) group together | |
| | with the carbon to which they are bound; | |
| | R₉ and R₁₀ are independently hydrogen; substituted or non- | |
| | substituted C₁₋C₁₂ alkyl; C₆₋C₁₀ aryl; a COR′′′ group (where R′′′ | |
| | is hydrogen; hydroxyl; substituted or non-substituted C₁₋C₁₂ alkyl; | |
| | substituted or non-substituted C₆₋C₁₀ aryl; O—C₁₋C₁₂ alkyl; or | |
| | N(R^{IV})(R^{V}) amino, where R^{IV} and R^{V} are independently hydrogen | |
| | or a C₁₋C₁₂ alkyl group); a (CH₂)n—OH carbinol group (where n is | |
| | an integer comprised between 1 and 10); or together form a | |
| | methylene group; | |
| | the bond [symbol] means a double bond or a single bond; and | |
| | where the tricyclic structure | |
| | 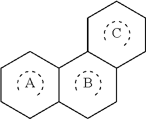 | |
| | is selected from the following structures | |
| | 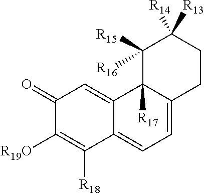 | (a) |
| | 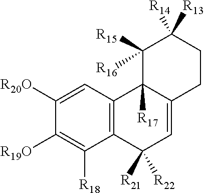 | (b) |
| | 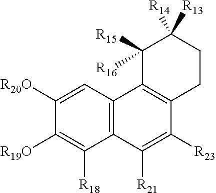 | (c) |

| | 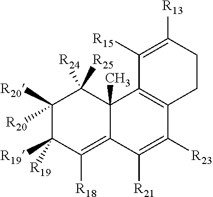 | |
|---|---|---|
| | 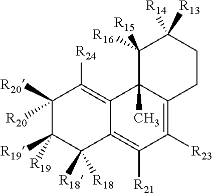 | |
| | 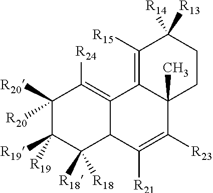 | |
| | wherein | |
| | R₁₃, R₁₄, R₁₅, R₁₆, R₂₁, R₂₂ and R₂₃ are independently hydrogen; | |
| | hydroxyl; halogen; substituted or non-substituted C₁₋C₁₂ alkyl; | |
| | substituted or non-substituted C₆₋C₁₀ aryl; a N (R^{VI})(R^{VII}) amino | |
| | group, where R^{VI} and R^{VII} are independently hydrogen or a C₁₋C₁₂ | |
| | alkyl group; an OCOR^{VIII} group, where R^{VIII} is (CH₂)₂COOH or | |
| | (CH₂)₂CO₂CH₂CH₃; or each pair can form a (C═O) group together | |
| | with the carbon to which they are bound or each pair can form a | |
| | (C═O) group together with the carbon to which they are bound; | |
| | R₁₇ is hydrogen or methyl; | |
| | R₁₈ and R₁₈′ are independently hydrogen; hydroxyl; halogen; C₁₋ | |
| | C₁₂ alkyl; C₆₋C₁₀ aryl; COR^{IX} (where R^{IX} is hydrogen; hydroxyl; | |
| | C₁₋C₁₂ alkyl; N(R^{X})(R^{XI}) amino, where R^{X} and R^{XI} are | |
| | independently hydrogen or a C₁₋C₁₂ alkyl group; or C₁₋C₁₂ | |
| | alkoxyl); or trifluoromethyl; | |
| | R₁₉, R₁₉′, R₂₀ and R₂₀′ are independently hydrogen; substituted or | |
| | non-substituted C₁₋C₁₂ alkyl; a COR^{XII} group (where R^{XII} is | |
| | hydrogen; hydroxyl; substituted or non-substituted C₁₋C₁₂ alkyl; | |
| | substituted or non-substituted C₆₋C₁₀ aryl; or N(R^{XIII})(R^{XIV}) amino, | |
| | where R^{XIII} and R^{XIV} are independently hydrogen or a C₁₋C₁₂ alkyl | |
| | group); a [(C₁-C₁₂)alkyl-O—(C₁-C₁₂)alkyl-]ₙ group (where n is | |
| | comprised between 1 and 3); trifluoromethyl; or each pair 19-19′ | |
| | or 20-20′ can form a group C═O together with the carbon to which | |
| | they are bound; | |
| | R₂₄ and R₂₅ are independently hydrogen, hydroxyl or halogen; | |
| | The preferred compounds which are in the previous structure are | |
| | selected from the group consisting of: | |
| | 3,9-dihydroxy-4,6b,8a,11,12b,14a-hexamethyl- | |
| | 7,8,8a,11,12,12a,12b,13,14,14a-decahydro-6bH,9H-picene- | |
| | 2,10-dione; | |
| | Acetic acid 9-hydroxy-4,6b,8a,11,12b,14a-hexamethyl-2,10- | |
| | dioxo-2,6b,7,8,8a,9,10,11,12,12a,12b,13,14,14a- | |
| | tetradecahydro-picen-3-yl ester; | |
| | Propionic acid 9-hydroxy-4,6b,8a,11,12b,14a-hexamethyl- | |
| | 2,10-dioxo-2,6b,7,8,8a,9,10,11,12,12a,12b,13,14,14a- | |
| | tetradecahydropicen-3-yl ester; | |
| | Dodecanoic acid 9-hydroxy-4,6b,8a,11,12b,14a-hexamethyl- | |
| | 2,10-dioxo-2,6b,7,8,8a,9,10,11,12,12a,12b,13,14,14a- | |
| | tetradecahydro-picen-3-yl ester; | |
| | Carbamic dimethyl acid 9-hydroxy-4,6b,8a,11,12b,14a- | |
| | hexamethyl-2,10-dioxo- | |
| | 2,6b,7,8,8a,9,10,11,12,12a,12b,13,14,14a-tetradecahydropicen- | |
| | 3-yl ester; | |
| | Nicotinic acid 9-hydroxy-4,6b,8a,11,12b,14a-hexamethyl- | |
| | 2,10-dioxo-2,6b,7,8,8a,9,10,11,12,12a,12b,13,14,14a- | |
| | tetradecahydro-picen-3-yl ester; | |
| | Benzoic acid 4-bromo-(9-hydroxy-6b,8a,11,12b,14a- | |
| | hexamethyl-2,10-dioxo- | |
| | 2,6b,7,8,8a,9,10,11,12,12a,12b,13,14,14a-tetradecahydropicen- | |
| | 3-yl)ester; | |
| | 14-bromo-3,7,9-trihydroxy-4,6b,8a,11,12b,14a-hexamethyl- | |
| | 7,8,8a,11,12,12a,12b,13,14,14a-decahydro-6bH,9H-picene- | |
| | 2,10-dione; | |
| | Carbamic dimethyl acid 12-bromo-9-hydroxy- | |
| | 6b,8a,11,12b,14a-hexamethyl-2,10-dioxo- | |
| | 2,6b,7,8,8a,9,10,11,12,12ar 12br 13,14,14a- | |
| | tetradecahydropicen-3-yl ester; | |
| | Benzoic acid 4-bromo-(12-bromo-9-hydroxy- | |
| | 6b,8a,11,12b,14a-hexamethyl-2,10-dioxo- | |
| | 2,6b,7,8,8a,9,10,11,12,12a,12b,13,14,14a-tetradecahydro- | |
| | picen-3-yl)ester; | |
| | 12-bromo-3,9-dihydroxy-6b,8a,11,12b,14a-hexamethyl- | |
| | 7,8,8a,11,12,12a,12b,13,14,14a-decahydro-6bHr9H-picene- | |
| | 2,10-dione; | |
| | 3,9,10-trihydroxy-6b,8a,11,12b,14a-hexamethyl- | |
| | 7,8,8a,9,10,11,12,12a,12b,13,14,14a-dodecahydro-6bH- | |
| | picene-2-one; | |
| | Succinic acid mono-(10-hydroxy-2,4ar 6ar,9, | |
| | 12b,14ahexamethyl-3,11-dioxo- | |
| | 1,2,3,4,4a,5,6,6a,11,12b,13,14,14a,14b-tetradecahydropicen-4- | |
| | yl)ester; | |
| | Succinic acid 10-hydroxy-2,4a,6a,9,12b,14a-hexamethyl-3,11- | |
| | dioxo-1,2,3,4,4a,5,6,6a,11,12b,13,14,14a,14b- | |
| | tetradecahydropicen-4-yl ester ethyl ester. | |
| | Carboxylic acid 7,10,11-trihydroxy-2,4a,6a,9,12b,14a- | |
| | hexamethyl-8-oxo-1,2,3,4,4a,5,6,6a,8,12b,13,14,14a,14b- | |
| | tetradecahydro-picene-2-methyl ester; | |
| | Carboxylic acid 9-formyl-10,11-dihydroxy-2,4a,6a,12b,14a- | |
| | pentamethyl-8-oxo-1,2,3,4,4a,5,6,6a,8,12b,13,14,14a,14b- | |
| | tetradecahydro-picene-2-methyl ester; | |
| | Carboxylic acid 11-hydroxy-10-(2-methoxy-ethoxymethoxy)- | |
| | 2,4a,6a,9,12b,14a-hexamethyl-8-oxo- | |
| | 1,2,3,4,4a,5,6,6a,8,12b,13,14,14a,14b-tetradecahydro-picene- | |
| | 2-methyl ester. | |
| IX | A compound as defined in international patent application | |
| | WO2007077203 having the general structure of the formula | |
| | 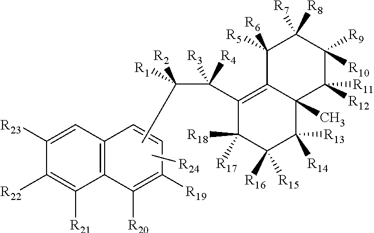 | |
| | wherein | |
| | R₁, R₂, R₃, R₄, R₅, R₆, R₉, R₁₀, R₁₁, R₁₂, R₁₃, R₁₄, R₁₅, R₁₆, R₁₇, R₁₈, | |
| | R₁₉ and R₂₀ are independently hydrogen; hydroxyl; halogen; | |
| | substituted or non-substituted C₁₋C₁₂ alkyl; substituted or non- | |
| | substituted C₆₋C₁₀ aryl; a N(R^{XV}) (R^{XVI}) amino group, where R^{XV} | |
| | and R^{XVI} are independently hydrogen or a C₁₋C₁₂ alkyl group; or | |
| | each pair can form a (C═O) carboxyl group together with the | |
| | carbon to which they are bound; | |
| | R₇ and R₈ are independently hydrogen; substituted or non- | |
| | substituted C₁₋C₁₂ alkyl; C₆₋C₁₀ aryl; a COR^{XVII} group (where | |
| | R^{XVII} is hydrogen; hydroxyl; substituted or non-substituted C₁₋C₁₂ | |
| | alkyl; substituted or non-substituted C₆₋C₁₀ aryl; O—C₁-C₁₂ alkyl; or | |
| | N(R^{XVIII})(R^{XIX}) amino, where R^{XVIII} and R^{XIX} are independently | |
| | hydrogen or a C₁₋C₁₂ alkyl group); a (CH2)ₙ—OH carbinol group | |
| | (where n is an integer comprised between 1 and 10); or together | |
| | form a methylene group, | |
| | R₂₁ and R₂₄ are independently substituted or non-substituted C₁₋ | |
| | C₁₂ alkyl; a COR^{XX} group (where R^{XX} is hydrogen; hydroxyl; | |
| | substituted or non-substituted C₁₋C₁₂ alkyl; substituted or non- | |
| | substituted C₆₋C₁₀ aryl; or N(R^{XXI})(R^{XXII}) amino, where R^{XXI} and | |
| | R^{XXII} are independently hydrogen or a C₁₋C₁₂ alkyl group); a [(C₁- | |
| | C₁₂)alkyl-O—(C₁-C₁₂ₐ)alkyl-]ₙ group (where n is comprised | |
| | between 1 and 3); or trifluoromethyl; | |
| | R₂₂ and R₂₃ are: | |
| | hydrogen; substituted or non-substituted C₁₋C₁₂ alkyl; a | |
| | COR^{XXIII} group (where R^{XXIII} is hydrogen; hydroxyl; | |
| | substituted or non-substituted C₁₋C₁₂ alkyl; substituted or non- | |
| | substituted C₆₋C₁₀ aryl; or N(R^{XXIV})(R^{XXV}) amino, where | |
| | R^{XXIV} and R^{XXV} are independently hydrogen or a C₁₋C₁₂ alkyl | |
| | group); a [(C₁-C₁₂)alkyl-O—(C₁-C₁₂ₐ)alkyl-]ₙ group (where n is | |
| | comprised between 1 and 3); or trifluoromethyl when R₂₄ is in | |
| | the para position with respect to R₂₀; or | |
| | OR₂₂′ and OR₂₃′ respectively, where R₂₂′ and R₂₃′ are | |
| | independently hydrogen; substituted or non-substituted C₁₋C₁₂ | |
| | alkyl; a COR^{XXVI} group (where R^{XXVI} is hydrogen; hydroxyl; | |
| | substituted or non-substituted C₁₋C₁₂ alkyl; substituted or non- | |
| | substituted C₆₋C₁₀ aryl; or N(R^{XXVII})(R^{XVIII}) amino), wherein | |
| | R^{XXVII} and R^{XVIII} are independently hydrogen or a C₁₋C₁₂ alkyl | |
| | group); a [(C₁-C₁₂)alkyl-O—(C₁-C₁₂ₐ)alkyl-]ₙ group (where n is | |
| | comprised between 1 and 3); or trifluoromethyl when R₂₄ is in | |
| | the meta position with respect to R₂₀. | |
| | The preferred compounds which are within the previous structure | |
| | are selected from the group of: | |
| | 14-bromo-3-hydroxy-4,6b,8a,11,12b,14a-hexamethyl- | |
| | 7,8,8a,11,12,12a,12b,13,14,14a-decahydro-6bH,9H-picene- | |
| | 2,10-dione; | |
| | Acetic acid 4,6b,8a,11,12b,14a-hexamethyl-2,10-dioxo- | |
| | 2,6b,7,8,8a,9,10,11,12,12a,12b,13,14,14a- | |
| | tetradecahydropicen-3-yl ester; | |
| | Nicotinic acid 4,6b,8a,11,12b,14a-hexamethyl-2,10-dioxo- | |
| | 2,6b,7,8,8a,9,10,11,12,12a,12b,13,14,14a- | |
| | tetradecahydropicen-3-yl ester; | |
| | 3,10-dihydroxy-4,6b,8a,11,12b,14a-hexamethyl- | |
| | 7,8,8a,9,10,11,12,12a,12b,13,14,14a-dodecahydro-6bHpicene- | |
| | 2-one; | |
| | 3-hydroxy-4,6b,8a,11,12b,14a-hexamethyl- | |
| | 7,8,8a,12a,12b,13,14,14a-octahydro-6bH,9H-picene-2,10- | |
| | dione; | |
| | 10,11-dihydroxy-2,4a,6a,9,14a-pentamethyl- | |
| | 1,4,4a,5,6,6a,13,14,14a,14b-decahydro-2H-picene-3-one; | |
| | 10,11-dihydroxy-2,4a,6a,9,14a-pentamethyl- | |
| | 4a,5,6,6a,13,14,14a,14b-octahydro-4H-picene-3-one. | |
| X | ATP analogs including non-hydrolysable ATP analogs such as AMP- | |
| | PCH₂P, adenylyl imidodiphosphate (AMP-PNP), AMP-PSP and AMP | |
| | where the oxygen bonding the second and third phosphates of the ATP | |
| | analogs is changed for CH₂, S (such as ATPγS, ATPβ and ATPαS) and | |
| | NH, respectively, as well as suicide substrates such as 5′-(p- | |
| | fluorosulfonyl benzoyl) adenosine (FSBA), N⁶-Diethyl-beta,gamma- | |
| | dibromomethylene-ATP, 2-methylthio-ATP (APM), α,β-methylene- | |
| | ATP, β,γ-methylene-ATP, di-adenosine pentaphosphate (Ap5A), 1,N⁶- | |
| | ethenoadenosine triphosphate, adenosine 1-oxide triphosphate, 2′ ,3′-O- | |
| | (benzoyl-4-benzoyl)-ATP (B-ZATP), the family of the ATP analogs | |
| | described in US2004204420, 2′,3′-O-(2,4,6-trinitrophenyl)-ATP (TNP- | |
| | ATP), 1-N⁶(methoxy)ATP, 7-N⁶-(pyrrolidine)ATP, 2-N⁶ (ethoxy) ATP, | |
| | 8-N⁶ (cyclopentyl) ATP, 3-N⁶(acetyl) ATP, 9-N⁶(cyclopentyloxy)ATP, | |
| | 4-N⁶ (i-propoxy) ATP, 10-N⁶-(Piperidine) ATP, 5-N⁶-(benzyl) ATP, 11- | |
| | N⁶-(cyclohexyl) ATP and the like. | |
| XI | Inhibitors of choline transporter such as analogs of N-n-alkylnicotinium, | |
| | HC-3 hemicholiniums, decamethonium, suxamethonium, D- | |
| | tubocurarine, tetramethylammonium, tetraethylammonium, | |
| | hexamethonium, N-alkyl analogs (N-ethyl choline, N-methyl choline), | |
| | mono-, di- and triethyl choline, N-hydroxyethyl pyrrolidinium methiodide | |
| | (pyrrolcholine), and DL-alpha-methyl choline described by Barker, L.A. and | |
| | Mittag, T. W. (J Pharmacol Exp Ther. 1975; 192: 86-94), dimethyl-n-pentyl | |
| | (2-hydroxyethyl) ammonium ion, decamethonium, hexamethonium | |
| | substituted with bis-catechol and decamethonium analogs described by Cai | |
| | et al. (Bioorganic & Medicinal Chemistry, 2007, 15: 7042-7047) having the | |
| | structure | |
| | 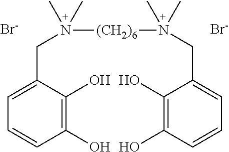 | 1 |
| | 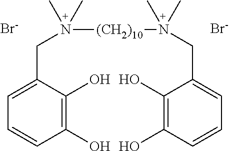 | 2 |

| | |
|---|---|
| XII | Inhibitor antibodies capable of binding specifically to and inhibiting the |
| | activity of choline kinase and, particularly, monoclonal antibodies which |
| | recognize the catalytic domain or the ChoKα dimerization domain and |
| | therefore inhibit the ChoKα activity. Examples of inhibitor antibodies are |
| | monoclonal antibodies as defined in WO2007138143. Other examples of |
| | inhibitor antibodies are the AD3, AD8 and AD11 antibodies as defined |
| | in WO2007138143. |
| XIII | Phosphatidylethanolamine N-methyltransferase (PEMT or EC 2.1.1.17) |
| | inhibitors. The treatment of cells with ChoKα inhibitors causes an increase |
| | in PEMT expression (Spanish patent application P200802007 co-pending |
| | with the present). Furthermore, the overexpression of ChoKβ in cells also |
| | causes an increase in the PEMT expression (Spanish patent application |
| | P200802007 co-pending with the present) suggesting that PEMT |
| | activation could be the pathway used by ChoKβ to compensate the |
| | decrease in the phosphatidylcholine levels in response to ChoKα |
| | inhibition. PEMT suitable for its use include 3-deazaadenosine (DZA) |
| | (Vance et al., 1986, Biochem. Biophys. Acta, 875: 501-509), 3- |
| | deazaaristeromycin (Smith and Ledoux, Biochim Biophys Acta. 1990, |
| | 1047: 290-3), bezafibrate and clofibric acid (Nishimaki-Mogami T et al., |
| | Biochim Biophys. Acta, 1996, 1304: 11-20). |
| XIV | An antisense oligonucleotide specific for the choline kinase sequence |
| XV | A DNA enzyme or ribozyme specific for the choline kinase sequence |
| XVI | An interfering RNA specific for the choline kinase sequence such as short |
| | hairpin RNA (shRNA) or the siRNA defined by Glunde et al. (Cancer Res., |
| | 2005, 65: 11034-11043). |
| XVII | Inhibitors of ChoKα capable of producing an increase in the expression |
| | levels of PEMT or ChoKβ proteins as defined in international patent |
| | application PCT/IB2009/052936 such as the chemical inhibitor MN58b. |

In a preferred embodiment, the therapy is a ChoKα inhibitor. In a more preferred embodiment, the ChoKα inhibitor is selected from table 2. In a still more preferred embodiment, the ChoKα inhibitor has the structure:

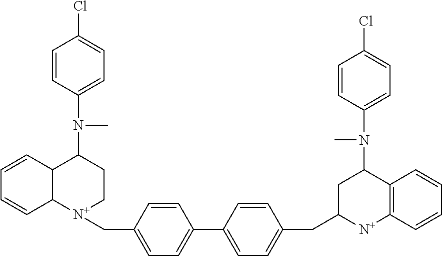 [SMILES: CN(c1ccc(Cl)cc1)C1CC(Cc2ccc(-c3ccc(C[NH+]4CCC(N(C)c5ccc(Cl)cc5)C5C=CC=CC54)cc3)cc2)[NH2+]c2ccccc21]

or a pharmaceutically acceptable salt or solvate thereof.

The term “pharmaceutically acceptable salt” as used herein, refers to salts that retain the biological effectiveness of the free acids and bases of the specified compound and that are not biologically or otherwise undesirable.

Examples of pharmaceutically acceptable salts include those salts prepared by reaction of the compounds described herein with a mineral or organic acid such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, ?-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

The term “solvate” describes a molecular complex comprising the compound and which further includes a stoichiometric or non-stoichiometric amount of solvent such as water, acetone, ethanol, methanol, dichloromethane, 2-propanol, or the like, bound by non-covalent intermolecular forces. In a particular case, the solvent us water, in which case the solvate is known as “hydrate”.

The term “pharmaceutically acceptable” as used herein, refers to a material, such as a carrier or diluent, which does not abrogate the biological activity or properties of the compounds described herein, and is relatively nontoxic, i.e, the material may be administered to an individual without causing undesirable biological effects or interacting in a deleterious manner with any of the components of the composition in which it is contained

The expression “folate antimetabolite” is used herein interchangeably with “folate antagonist” and refers to a compound that inhibits the activity of at least one folate-dependent enzyme. By a “folate-dependent enzyme,” it is intended an enzyme which requires folate or a folate metabolite to perform at least one of its catalytic activities. In some embodiments, the folate antagonist inhibits the activity of at least one folate-dependent enzyme selected from dihydrofolate reductase (EC 1.5.1.3), folylpolyglutamate synthetase (EC 6.3.2.17), glycinamide ribonucleotide formyltransferase (EC 2.1.2.2), aminoimidazole carboxamide ribonucleotide formyltransferase (EC 5.3.1.16), and thymidylate synthase (EC 2.1.1.45).

Suitable folate antagonists include, without limitation, DHFR inhibitors such as methothrexate, trimetrexate and edatrexate; TS inhibitors such as raltitrexed, pemetrexed, GW1843, OSI-7904L, nolatrexed and ZD9331; and the GART inhibitors lomotrexol, and LY309887 (Purcell and Ettinger (2003) Current Oncology Reports 4:114-25). In a preferred embodiment, the folate antagonist is pemetrexed.

The term “EGFR-targeted drug”, as used herein has been described above in detail and is used in the present method with the same meaning.

The term “antimicrotubule agent”, as used herein, refers to an agent which interferes with cell division by disrupting the normal functionality of the cellular microtubules. Exemplary antimicrotubule agents may include, but are not limited to, taxanes, such as taxol and taxotere, and vinca alkaloids, such as vincristine and vinblastine.

In the context of the second method of the invention, the term “subject” is understood as a subject suffering from cancer who has not received or is not receiving a chemotherapeutic treatment. In a preferred embodiment the subject is as subject suffering from NSCLC who has not received or is not receiving a platinum-based chemotherapeutic treatment.

The skilled person will appreciate that the particular embodiments developed in the first method of the invention are also applicable to the second method of the invention, such as (i) the type of NSCLC (squamous cell carcinoma of the lung, large cell carcinoma of the lung or adenocarcinoma of the lung), (ii) the stage of the NSCLC (Stage IIIA, IIIB or IV), (iii) the kind of sample obtained from the subject (tissue sample, preferably a tumour tissue sample, more preferably a lung tumour tissue sample), (iv) the different procedures for determining the expression levels of ChoKα gene (measuring the levels of mRNA or protein or variants thereof encoded by said gene), (v) the method to determine the mRNA expression levels, preferably by quantitative PCR, more preferably by Real-Time PCR, (vi) the method to determine the ChoKα expression levels, preferably by Western blot or immunohistochemistry or (vii) the platinum-based chemotherapeutic treatments used in the chemotherapy (carboplatin, cisplatin, oxaliplatin and the combinations TCGV, CGV and CI-TA). Moreover, the skilled person will also understand that all methods and techniques previously cited for determining the protein and mRNA expression levels can also be used in the second method of the invention.

### Kits of the Invention and Uses Thereof

In another aspect, the invention relates to the use of a reagent capable of determining the expression levels of ChoKα gene in a sample from a subject suffering from cancer for predicting the clinical response of said subject to a chemotherapeutic treatment.

In another aspect, the invention relates to the use of a reagent capable of determining the expression levels of ChoKα gene in a sample from a patient for predicting the clinical response or the lack of clinical response of said patient to a therapy selected from the group consisting of:

(i) a ChoKα inhibitor,

(ii) a folate antimetabolite,

(iii) an antimicrotubule agent,

(iv) an EGFR-targeted drug,

(v) a combination of one or more of (i) to (iv) above

In a preferred embodiment the reagent is capable of determining the expression levels of ChoKα gene in a sample from a subject suffering from NSCLC for predicting the clinical response of said subject to a platinum-based chemotherapeutic treatment.

In a preferred embodiment, the reagent is adequate for determining the expression levels of the ChoKα a isoform. In another preferred embodiment, the reagent is adequate for determining the expression levels of both ChoKα a and b isoforms. In another preferred embodiment, the reagent is adequate for determining the expression levels of the ChoKα b isoform. In a preferred embodiment, the reagent is adequate for determining the expression levels of the ChoKα a isoform but is not adequate for determining the expression levels of the b isoform. In another preferred embodiment, the reagent is adequate for determining the expression levels of the ChoKα b isoform but is not adequate for determining the expression levels of the ChoKα a isoform.

In another aspect, the invention relates to the use of a reagent capable of determining the expression levels of ChoKα gene in a sample from a subject suffering from cancer for designing and individual therapy for a subject suffering from said cancer. In a preferred embodiment the subject is suffering from NSCLC. In a preferred embodiment, the reagent is adequate for determining the expression levels of the ChoKα a isoform. In another preferred embodiment, the reagent is adequate for determining the expression levels of both ChoKα a and b isoforms. In another preferred embodiment, the reagent is adequate for determining the expression levels of the ChoKα b isoform. In a preferred embodiment, the reagent is adequate for determining the expression levels of the ChoKα a isoform but is not adequate for determining the expression levels of the b isoform. In another preferred embodiment, the reagent is adequate for determining the expression levels of the ChoKα b isoform but is not adequate for determining the expression levels of the ChoKα a isoform.

In a preferred embodiment the clinical response is measured as time to progression or progression-free survival.

The term “reagent”, as used herein, refers to any compound or composition which can be used for detecting ChoKα gene or for detecting ChoKα protein or variants thereof and, optionally, reagents for detecting one or more housekeeping genes or the protein encoded by said housekeeping gene(s). This set of reagents can include, without limitation, nucleic acids capable of specifically hybridising with the ChoKα gene and/or antibodies or fragments thereof capable of specifically binding to ChoKα protein or to variants thereof (including fragments thereof containing antigenic determinants).

The reagents for use in the method of the invention may be formulated as a “kit” and thus, may be combined with one or more other types of elements or components (e.g., other types of biochemical reagents, containers, packages such as packaging intended for commercial sale, substrates to which the reagents are attached, electronic hardware components, etc.).

In a preferred embodiment, the reagents for determining the expression levels of ChoKα gene are probes, primers and/or antibodies.

Nucleic acids capable of specifically hybridizing with the ChoKα gene are, for example, one or more pairs of primer oligonucleotides for the specific amplification of fragments of the mRNA (or of their corresponding cDNA) of said gene and/or one or more probes for the identification of this gene.

As the skilled person understands, the oligonucleotide primers and probes of the kit of the invention can be used in all techniques of gene expression profiling (RT-PCR, SAGE, TaqMan, Real Time-PCR, FISH, NASBA, etc).

Antibodies, or a fragment thereof, capable of detecting an antigen, capable of specifically binding to ChoKα protein or to variants thereof are, for example, monoclonal and polyclonal antibodies, antibody fragments, Fv, Fab, Fab′ and F(ab′)2, ScFv, diabodies, triabodies, tetrabodies and humanised antibodies. The antibodies of the kit of the invention can be used in conventional methods for detecting protein expression levels, such as Western-blot or Western transfer, ELISA (enzyme linked immunosorbent assay), RIA (radioimmunoassay), competitive EIA (enzymatic immunoassay), DAS-ELISA (double antibody sandwich ELISA), immunocytochemical and immunohistochemical techniques, techniques based on the use of biochips, protein microarrays including specific antibodies or assays based on colloidal precipitation in formats such as dipsticks, etc.

Said reagents, specifically the probes and the antibodies, may be fixed onto a solid support, such as a membrane, a plastic or a glass, optionally treated in order to facilitate fixation of said probes or antibodies onto the support. Said solid support, which comprises, at least, a set of antibodies capable of specifically binding to ChoKα protein or to variants thereof, and/or probes specifically hybridized with the ChoKα gene, may be used for the detection of the expression levels by means of array technology.

The kits of the invention optionally comprise additional reagents for detecting a polypeptide encoded by a housekeeping gene or the mRNA encoded by said housekeeping gene. The availability of said additional reagent allows the normalization of measurements taken in different samples (e.g. the test sample and the control sample) to exclude that the differences in expression of the biomarker are due to a different amount of total protein in the sample rather than to real differences in relative expression levels. Housekeeping genes, as used herein, relates to genes which code for proteins which are constitutively expressed and carry out essential cellular functions. Preferred housekeeping genes for use in the present invention include β-2-microglobulin, ubiquitin, 18-S ribosomal protein, cyclophilin, PSMB4, GAPDH, tubulin and β-actin.

The terms “ChoKα inhibitor”, “folate antimetabolite”, “antimicrotubule agent” and “EGFR-targeted drug” have been described above in the context of the methods for designing an individualised therapy for a cancer patient and are equally applied in the present aspect.

All the particular embodiments disclosed for the methods of the present invention are applicable to the kit of the invention and uses thereof.

### Therapeutic Methods of the Invention

The results obtained in the present invention show that high levels of expression of ChoKα in advanced NSCLC tumours indicates a high probability of a non-successful chemotherapeutic treatment with cisplatin-based treatments. On the other hand low or equal levels of ChoKα are indicative of a higher probability of a better response to said treatment, an indication that this may be the best treatment available for such patients. ChoKα inhibitors would be the choice as first line treatment for patients with high levels of expression of ChoKα. Alternative treatments for cisplatin-based regimens in these NSCLC patients include pemetrexed or tyrosine kinase inhibitors such as Tarceva or Iressa. Thus, high levels of expression of ChoKα would indicate that these established alternative treatments or other to be developed in the future are first line treatments.

In another aspect, the invention relates to a platinum-based chemotherapeutic treatment for use in the treatment of NSCLC in a subject, wherein a sample of said subject shows low or equal expression levels of ChoKα gene with respect to reference values.

Alternatively, the invention relates to the use of a platinum-based chemotherapeutic treatment for the manufacture of a medicament for the treatment of a subject suffering from NSCLC, wherein the subject shows low or equal expression levels of ChoKα gene with respect to reference values.

In another aspect, the invention relates to a method for the treatment of NSCLC in a subject comprising administering to said subject a platinum-based chemotherapeutic treatment wherein the subject shows low or equal expression levels of ChoKα gene with respect to reference values.

In a preferred embodiment, the subject shows low or equal expression levels of the ChoKα a isoform. In another preferred embodiment, the subject shows low or equal expression levels of both a and b isoforms of ChoKα. In another preferred embodiment, the subject shows low or equal expression levels of the ChoKα b isoform.

Platinum-based chemotherapeutic treatments for use in the treatment of a subject suffering from NSCLC are broadly known from the state of the art and have been previously described herein. The chemotherapeutic treatment may include single platinum-based compounds as well as combinations comprising platinum compounds such as paclitaxel followed by cisplatin-gemcitabine-vinorelbine, cisplatin-gemcitabine-vinorelbine and cisplatin and docetaxel. In a preferred embodiment, the platinum-based chemotherapeutic treatment is Taxol® (paclitaxel) followed by cisplatin-gemcitabine-vinorelbine (T-CGV regimen), cisplatin-gemcitabine-vinorelbine (CGV regimen), and cisplatin-Taxotere® (docetaxel) (CI-TA regimen).

In another aspect, the invention relates to a ChoKα inhibitor, a folate antimetabolite, an antimicrotubule agent, an EGFR-targeted drug or a combination of one or more of the above for use in the therapy of a subject suffering from NSCLC, wherein a sample of said subject shows high expression levels of ChoKα gene with respect to reference values.

Alternatively, the invention relates to the use of a ChoKα inhibitor, of a folate inhibitor, of a antimicotubule agent, of an EGFR-targeted drug or of a combination of one or more of the above for the manufacture of a medicament for the treatment of NSCLC, wherein the subject shows high expression levels of ChoKα gene with respect to reference values.

Alternatively, the invention relates to a method for the treatment of NSCLC in a subject comprising the administration to said subject of a ChoKα inhibitor, a folate antimetabolite, an antimicrotubule agent, an EGFR-targeted drug or of a combination of one or more of the above, wherein the subject shows high expression levels of ChoKα gene with respect to reference values.

The terms “chemotherapeutic treatment”, “subject”, “NSCLC”, “reference values”, “ChoKα inhibitor”, “folate inhibitor”, “antimicrotubule agent”, and “EGFR-targeted drug” have already been explained in the part of the description related to the other methods of the invention.

### Method for the Identification of a Patient Likely to Respond to a Therapy

The inventors of the present invention have discovered that, surprisingly, the expression levels of the ChoKα gene are useful for the identification of patients likely to respond to a therapy

Thus, in one aspect, the invention relates to an in vitro method for the identification of a patient likely to respond to a therapy selected from the group consisting of:

(i) a choline kinase alpha (ChoKα) inhibitor,

(ii) a folate antimetabolite,

(iii) an antimicrotubule agent,

(iv) an EGFR-targeted drug and

(v) a combination of one or more of (i) to (iv) above

comprising determining the expression level of ChoKα gene in a sample of said patient and comparing said level with a reference value,
wherein an increase in the expression level of ChoKα gene in said sample with respect to said reference value is indicative that the patient is likely to respond to said therapy or wherein a decrease or lack of change in the expression level of ChoKα gene in said sample with respect to said reference value is indicative that the patient is unlikely to respond to said therapy.

The terms “patient”, “cancer”, “choline kinase alpha”, “a choline kinase alpha (ChoKα) inhibitor”, “a folate antimetabolite”, “an antimicrotubule agent”, “an EGFR-targeted drug”, “the expression level of ChoKα gene”, “sample”, “patient”, “response”, “reference value”, “increased expression levels”, “decreased expression levels”, “lack of change of expression” have been defined above in detail and are used in the same manner in the present aspect of the invention.

The method according to the present invention comprises in a first step the determination of the expression level of ChoKα gene in a sample of the cancer patient and the comparison of the expression levels with a reference value. In a preferred embodiment, the method of the invention comprises the determination of the expression levels of the ChoKα a isoform. In another preferred embodiment, the method of the invention comprises the determination of the expression levels of both ChoKα a and b isoforms. In another preferred embodiment, the method of the invention comprises the determination of the expression levels of the ChoKα b isoform. In a preferred embodiment, the method of the invention comprises the determination of the expression levels of the ChoKα a isoform but does not comprise the determination of the expression levels of the b isoform. In another preferred embodiment, the method of the invention comprises the determination of the expression levels of the ChoKα b isoform but does not comprise the determination of the expression levels of the a isoform.

In a preferred embodiment, the expression levels of the Chokα gene (or of the isoforms) are determined by measuring the levels of mRNA encoded by the ChoKα gene (or of the transcripts of each isoform). In a more preferred embodiment, the mRNA expression levels of the Chokα gene (or of the isoforms) are determined by quantitative PCR, preferably, Real-Time PCR.

In another embodiment, the expression levels of the Chokα gene are determined by determining the levels of ChoKα protein or of variants thereof. In a more preferred embodiment, the expression levels of ChoKα protein or of variants thereof are determined by Western blot or immunohistochemistry.

The term “reference value” has been explained in detail above and is used with the same meaning in the present method.

In a preferred embodiment, the therapy is a ChoKα inhibitor. In a more preferred embodiment, the ChoKα inhibitor is selected from table 2. In a still more preferred embodiment, the ChoKα inhibitor has the structure:

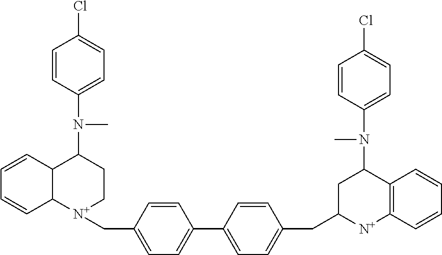 [SMILES: CN(c1ccc(Cl)cc1)C1CC(Cc2ccc(-c3ccc(C[NH+]4CCC(N(C)c5ccc(Cl)cc5)C5C=CC=CC54)cc3)cc2)[NH2+]c2ccccc21]

or an analog, salt or solvate thereof.

In an embodiment, the patient suffers from cancer. In a preferred embodiment, the cancer is selected from the group consisting of lung cancer, breast cancer, colon cancer, bladder cancer and pancreas cancer. In yet another embodiment, the lung cancer is non-small cell lung cancer (NSCLC). In a still more preferred embodiment, the the NSCLC is selected from squamous cell carcinoma of the lung, large cell carcinoma of the lung and adenocarcinoma of the lung. In another embodiment, the NSCLC is advanced stage NSCLC, preferably, stage IIIA, IIIB or IV.

In a preferred embodiment, the sample is a tissue sample, preferably a tumour tissue sample, more preferably a lung tumour tissue sample.

The following example is provided as merely illustrative and is not to be construed as limiting the scope of the invention.

### Example

### I. Material and Methods

### Patients

Patients were retrospectively collected at La Paz Hospital in Madrid (Spain) between 2001 and 2008. Inclusion criteria for this pilot study were patients who had primary NSCLC Stages III to IV, who were 18 years or older, and had received platinum-based chemotherapy as initial treatment modality. Exclusion criteria were patients who have previous treatment with either chemotherapy or radiotherapy and patients who could not be assessed for response. Only those specimens with a pathological analysis that included at least 80% of tumour in the paraffin-embedded tissue were included in the study. In total, the paraffin-embedded tumour lung tissue specimens from 30 patients who met the above criteria were retrospectively investigated.

Systemic chemotherapy using cis-diamminedichloroplatinum (CDDP) was performed in all patients. Regarding chemotherapeutic regimens used, Taxol® (paclitaxel) followed by cisplatin-gemcitabine-vinorelbine (T-CGV regimen) or just cisplatin-gemcitabine-vinorelbine (CGV regimen) were the most common options (73%). Some patients followed cisplatin-Taxotere® (docetaxel) (CI-TA regimen) that was administered to 26% of patients.

### Study Design

The present study was a retrospective analysis of the value of ChoKα mRNA expression to predict the response to platinum-based chemotherapy in patients with advanced NSCLC.

The standard response criteria were used to evaluate response to chemotherapy.

Response was defined by a reduction of >50% in the sum of products of the largest perpendicular diameters of all tumour localizations, with no new tumour lesions. Stabilization was defined by a <50% decrease or a <25% increase in tumour size. Progression was defined as an increase in the size of tumour lesions by >25% or appearance of new lesions.

Patient's response was classified in two groups, clinical benefit (including response and stabilization) and progression. Follow-up was performed according to the criteria used in the Thoracic Surgery Department, La Paz University Hospital, and included clinical assessments and CT of thorax every 3 months.

### Gene Expression Analysis

mRNA concentrations extracted from tissue samples were measured by use of quantitative RT-PCR.

Quantification of gene expression (AQ) was calculated with the 2^{-ΔCt} method and presented as AQ⁻¹⁰. Gene expression analysis was performed using 3 different endogenous genes for normalization (GAPDH, β-actin and PSMB4) obtaining similar results. Data presented here was analyzed using the well-established gene GAPDH for normalization, but similar results were obtained using the other endogenous genes.

### Statistical Analyses

Time to progression was used for the analysis of progression free-survival.

Receiver operating characteristic (ROC) curves were obtained to show the relationship between sensitivity and false-positive rate at different cut-off values of ChoKα expression for time to progression, and the cut-off value was established according to the best combination of sensitivity and false-positive rate (1-specificity) based on the ROC curves.

The Kaplan-Meier method was used to estimate progression-free survival.

The effect of the different factors on tumour progression was assessed by the log-rank test for univariate analysis. Hazard ratios (HR) and 95% confidence intervals (95% Cl) were calculated from the Cox regression model.

All reported p values were two-sided. Statistical significance was defined as p<0.05. Statistical analyses were done using the SPSS software (version 14.0).

### II. Results

### Patient Characteristics and Clinical Outcome

30 patients with Stages III and IV NSCLC were included in this study with a median lung-cancer-specific survival time of 11 months (95% CI: 6.7-15.3). Tumour progression was identified in 11 patients (37%), of which all of them died of lung cancer. The overall clinical benefit rate was 19/30 (63%), from which the response rate was 14/30 (47%), and 5 patients (17%) showed stable disease. Pathological and clinical parameters of patients included in the study are summarized in Table 3.

TABLE 3

| Patients | n (%) |
|---|---|
| Sex | |
| Men | 30 (100) |
| Tumour type | |
| Squamous-cell carcinoma | 17 (56.7) |
| Adenocarcinoma | 4 (13.3) |
| Large cell | 9 (30) |
| Tumour stage | |
| IIIB | 10 (33.3) |
| IV | 20 (66.7) |
| Chemotherapy | |
| CGV | 9 (30) |
| CI-TA | 8 (26.7) |
| T-CGV | 13 (43.3) |
| Response | |
| Response | 14 (46.7) |
| Stabilization | 5 (16.7) |
| Progression | 11 (36.7) |
| Exitus | |
| No | 5 (16.7) |
| Yes | 25 (83.3) |

### Gene Expression and Response to the Treatment

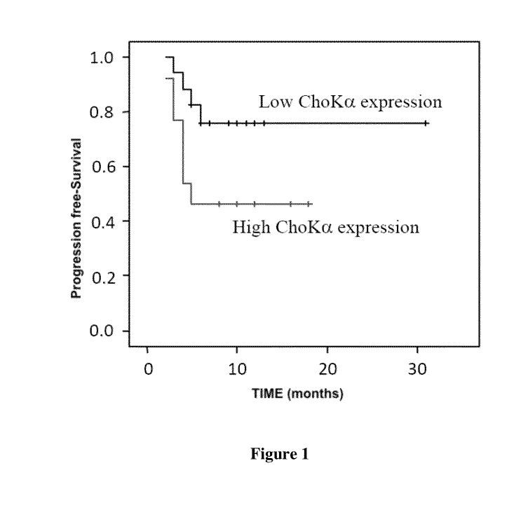

ChoKα mRNA concentrations were measured by use of quantitative RT-PCR using the Taqman probe with accession number Hs00608045_m1 and/or Taqman probe with accession number Hs03682798_m1. Gene-expression analysis showed that ChoKα expression was distributed differentially in the tumours, with normalised AQ values of mRNA copies oscillating between 0.07 and 15.44. According to ROC methodology, an arbitrary cut-off point of 1.784 AQ was established (64% sensibility, 68% specificity). Under these conditions, 13 out of the 30 (43%) tumour samples analysed for ChoKα overexpression were above this cut-off. Among the 19 patients who had clinical benefit from the treatment, 13 (68.4%) displayed low levels of ChoKα. By contrast, 7 out of the 11 patients with progressive disease (63.6%) displayed levels of ChoKα over the cut-off level. Accordingly, these patients with increased levels of ChoKα had worse progression-free survival than those with lower concentrations of this enzyme. Median progression-free survival was 5 months in patients with ChoKα expression above the cut-off level, whereas it was not reached at the time of assessment in those patients who had ChoKα expression below the cut-off level (p=0.05) (FIG. 1).

Univariate analysis of the prognostic significance of ChoKα expression showed that higher concentrations of the enzyme correlated with an increased risk of treatment failure compared with lower concentrations of ChoKα expression (p<0.05, HR 2.57 [95% IC: 0.69-9.56]).

### III. Discussion

The present invention explored the predictive value of ChoKα expression in tumour samples of subjects with advanced NSCLC receiving platinum-based chemotherapy regimens. This study strongly suggests that ChoKα over-expression is associated with poor response to platinum-based chemotherapy in subjects with advanced NSCLC. In addition, these results provide new insights for the biological properties and clinical relevance of ChoKα in NSCLC, rendering further evidence for the multifunctional effect of this enzyme in the onset and progression of the disease.

A significant association of ChoKα expression with treatment failure has been found, suggesting a promising value of this marker for the analysis of the evolution after treatment of subjects with advanced NSCLC. Thus, these results suggest that the prognosis of subjects with high expression of ChoKα would be poor after chemotherapy with platinum, which plays a central role in the management of NSCLC.

Concurrent chemotherapy with platinum is usually associated with significant toxicity. Therefore, it is reasonable to consider as appropriate and desirable the implementation of an alternative treatment strategy for subjects with high expression of this enzyme. Taking into account that ChoKα specific inhibitors have demonstrated efficient antitumoral activity both in vitro and in vivo (Ramírez de Molina A, et al. 2007, Lancet Oncol. vol. 8(10): 889-97; Lacal J C. 2001, IDrugs. vol. 4(4):419-26; Hernandez-Alcoceba R, et al. 1999, Cancer Res vol. 59: 3112-8; Ramirez de Molina A, et al. 2004, Cancer Res. vol. 64:6732-9), these results seem to be even more promising regarding their clinical implications. Furthermore, a synergistic effect has been observed under diverse experimental conditions when ChoKα inhibitors are combined with cisplatin in tumour cells derived from lung tumour (International patent application published as WO2010031825). Therefore, subjects that are resistant to cisplatin could be treated with a combination of ChoKα inhibitors and cisplatin.

Thus, the present invention shows a clear potential for a predictive value of ChoKα expression for response to platinum-based chemotherapy in subjects with advanced NSCLC and for the identification of subjects susceptible of alternative treatments to improve the clinical outcome.

1.-41. (canceled)

42. An in vitro method for predicting the clinical response of a subject suffering from cancer to a chemotherapeutic treatment comprising determining the expression level of choline kinase alpha (ChoKα) gene in a sample from the subject.

43. The method according to claim 42, wherein the method further comprises comparing the expression level of ChoKα with a reference value, wherein an alteration in the expression level of ChoKα gene in said sample with respect to said reference value is indicative of a poor clinical response of the subject to said chemotherapeutic treatment or of a good clinical response of the subject to said chemotherapeutic treatment.

44. The method according to claim 43, wherein the alteration in the expression levels of ChoKα is an increase in said expression level with respect to said reference value and wherein the increase in said expression level is indicative of a poor clinical response or wherein the alteration in the expression levels of ChoKα is an decrease in said expression level with respect to said reference value and wherein the decrease in said expression level is indicative of a good clinical response.

45. The method according to claim 42, wherein the chemotherapeutic treatment is selected from the group consisting of:
(i) a ChoKα inhibitor,
(ii) a folate antimetabolite,
(iii) an antimicrotubule agent,
(iv) an EGFR-targeted drug and
(v) a combination of one or more of (i) to (iv) above.

46. The method according to claim 45, wherein the ChoKα inhibitor is selected from Table 2.

47. The method according to claim 46, wherein the ChoKα inhibitor has the structure: 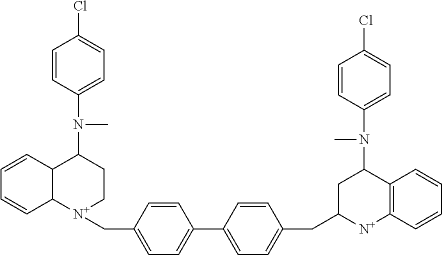 [SMILES: CN(c1ccc(Cl)cc1)C1CC(Cc2ccc(-c3ccc(C[NH+]4CCC(N(C)c5ccc(Cl)cc5)C5C=CC=CC54)cc3)cc2)[NH2+]c2ccccc21]
or a pharmaceutically acceptable salt or solvate thereof.

48. The method according to claim 42, wherein the cancer is selected from the group consisting of lung cancer, breast cancer, colon cancer and pancreas cancer.

49. The method according to claim 48, wherein the lung cancer is non-small cell lung cancer (NSCLC).

50. The method according to claim 49 wherein the NSCLC is selected from squamous cell carcinoma of the lung, large cell carcinoma of the lung, and adenocarcinoma of the lung.

51. The method according to claim 50, wherein the NSCLC is advanced stage NSCLC comprising stage IIIA, IIIB or IV NSCLC.

52. The method according to claim 42, wherein the sample comprises a tissue sample.

53. The method according to claim 42, wherein the expression levels of the ChoKα gene are determined by measuring the levels of mRNA encoded by the ChoKα gene, or the levels of ChoKα protein or of variants thereof.

54. The method according to claim 53, comprising at least one of: determining the mRNA expression levels by quantitative PCR, preferably, Real-Time PCR, and determining the expression levels of ChoKα protein or of variants thereof by Western blot or immunohistochemistry.

55. An in vitro method for designing an individual therapy for a subject suffering from cancer, comprising determining the expression level of choline kinase alpha (ChoKα) gene in a sample from the subject.

56. The in vitro method according to claim 55, wherein the method further comprises comparing the expression level of ChoKα with a reference value,
wherein a decrease or a lack of change in the expression level of ChoKα gene in said sample with respect to said reference value is indicative that the subject is a candidate for a therapy based on said chemotherapeutic treatment, or
wherein an increase in the expression level of ChoKα gene in said sample with respect to said reference value is indicative that the subject is a candidate for the treatment with a therapy selected from the group consisting of:
(i) a ChoKα inhibitor,
(ii) a folate antimetabolite,
(iii) an antimicrotubule agent,
(iv) an EGFR-targeted drug,
(v) a combination of one or more of (i) to (iv) above.

57. The in vitro method according to claim 55, wherein the chemotherapeutic treatment comprises a platinum-based chemotherapeutic treatment.

58. The in vitro method according to claim 55, wherein the cancer is non-small cell lung cancer (NSCLC).

59. The in vitro method according to claim 58, wherein the NSCLC is selected from squamous cell carcinoma of the lung, large cell carcinoma of the lung, and adenocarcinoma of the lung.

60. The in vitro method according to claim 58, wherein the NSCLC is advanced stage NSCLC comprising stage IIIA, IIIB or IV NSCLC.

61. The in vitro method according to claim 55, wherein the sample comprises a tissue sample.

62. A method for the treatment of NSCLC in a subject, comprising the administration of a platinum-based chemotherapeutic to said subject wherein a sample of said subject shows low or equal expression levels of ChoKα gene with respect to reference values, or the administration to said subject of a ChoKα inhibitor, a folate antimetabolite, an EGFR-targeted drug or a combination of one or more of the above, wherein a sample of said subject shows high expression levels of ChoKα gene with respect to reference values.

63. An in vitro method for the identification of a patient likely to respond to a therapy selected from the group consisting of:
(i) a choline kinase alpha (ChoKα) inhibitor,
(ii) a folate antimetabolite,
(iii) an antimicrotubule agent,
(iv) an EGFR-targeted drug and
(v) a combination of one or more of (i) to (iv) above
comprising determining the expression level of ChoKα gene in a sample of said patient and comparing said level with a reference value,
wherein an increase in the expression level of ChoKα gene in said sample with respect to said reference value is indicative that the patient is likely to respond to said therapy, or
wherein a decrease or lack of change in the expression level of ChoKα gene in said sample with respect to said reference value is indicative that the patient is unlikely to respond to said therapy.
